SEQUENCE LISTING (36 sequences)

SEQ ID NO: 1 - protein, 1074 aa, Homo Sapiens
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 2 - protein, 492 aa, Homo Sapiens
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVV

SEQ ID NO: 3 - protein, 282 aa, Homo Sapiens
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESG

SEQ ID NO: 4 - protein, 5 aa, Homo Sapiens
EPKSC

SEQ ID NO: 5 - protein, 5 aa, Homo Sapiens
EPKSS

SEQ ID NO: 6 - protein, 5 aa, Artificial Sequence
  linker
GGGGG

SEQ ID NO: 7 - protein, 5 aa, Artificial Sequence
  linker
GGGGS

SEQ ID NO: 8 - protein, 10 aa, Artificial Sequence
  linker
GGGGSGGGGS

SEQ ID NO: 9 - protein, 15 aa, Artificial Sequence
  linker
GGGGSGGGGSGGGGS

SEQ ID NO: 10 - protein, 20 aa, Artificial Sequence
  linker
GGGGSGGGGSGGGGSGGGGS

SEQ ID NO: 11 - protein, 25 aa, Artificial Sequence
  linker
GGGGSGGGGSGGGGSGGGGSGGGGS

SEQ ID NO: 12 - protein, 30 aa, Artificial Sequence
  linker
GGGGSGGGGSGGGGSGGGGSGGGGSGGGGS

SEQ ID NO: 13 - protein, 35 aa, Artificial Sequence
  linker
GGGGSGGGGSGGGGSGGGGSGGGGSGGGGSGGGGS

SEQ ID NO: 14 - protein, 226 aa, Homo Sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 508 aa, Artificial Sequence
  PDGFRbeta-D1-D3-hIgG1-Fc fusion protein  - polypeptide
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 513 aa, Artificial Sequence
  PDGFRbeta-D1-D3-1xGGGGS-hIgG1-Fc fusion polypeptide
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 518 aa, Artificial Sequence
  PDGFRbeta-D1-D3-2xGGGGS-hIgG1-Fc fusion polypeptide
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 523 aa, Artificial Sequence
  PDGFRbeta-D1-D3-3xGGGGS-hIgG1-Fc fusion polypeptide
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 528 aa, Artificial Sequence
  PDGFRbeta-D1-D3-4xGGGGS-hIgG1-Fc fusion polypeptide
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 533 aa, Artificial Sequence
  PDGFRbeta-D1-D3-5xGGGGS-hIgG1-Fc fusion polypeptide
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 539 aa, Artificial Sequence
  PDGFRbeta-D1-D3-6xGGGGS-hIgG1-Fc fusion polypeptide
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGGGGGSGGGGSGGGGSGGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 543 aa, Artificial Sequence
  PDGFRbeta-D1-D3-7xGGGGS-hIgG1-Fc fusion polypeptide
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGGGGGSGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 512 aa, Artificial Sequence
  PDGFRbeta-D1-D3-EPKSC-hIgG1-Fc fusion polypeptide
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 512 aa, Artificial Sequence
  PDGFRbeta-D1-D3-EPKSS-hIgG1-Fc fusion polypeptide
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 512 aa, Artificial Sequence
  PDGFRbeta-D1-D3-GGGG-hIgG1-Fc fusion polypeptide
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - DNA, 1536 bp, Artificial Sequence
  polynucleotide encoding PDGFRbeta-D1-D3-hIgG1-Fc
ctggtcgtgacacctcctggcccagaactggtgctgaacgtgtccagcaccttcgtgctgacctgcagcggatctgcccctgtcgtgtgggagcggatgagccaggaaccccctcaggaaatggccaaggcccaggacggcaccttcagcagcgtgctgacactgaccaacctgaccggcctggataccggcgagtacttctgcacccacaacgacagcagaggcctggaaaccgatgagcggaagcggctgtacatcttcgtgcccgatcctaccgtgggcttcctgcccaacgatgccgaggaactgttcatcttcctgaccgagattaccgagatcaccatcccctgcagagtgaccgacccccagctggtcgtgaccctgcacgagaagaaaggcgacgtggccctgcccgtgccctacgatcatcagagaggcttcagcggcatcttcgaggacagaagctacatctgcaagaccaccatcggcgacagagaggtggacagcgacgcctactacgtgtaccggctgcaggtgtcctccatcaacgtgtccgtgaacgccgtgcagaccgtcgtgcggcagggcgagaatatcaccctgatgtgcatcgtgatcggcaacgaggtcgtgaacttcgagtggacctacccccggaaagaaagcggcagactggtggaacccgtgaccgacttcctgctggacatgccctaccacatccggtccatcctgcacatccctagcgccgagctggaagatagcggcacctacacctgtaacgtgaccgagagcgtgaacgaccaccaggacgagaaggccatcaacatcaccgtggtggaaagcggccccaagagctgcgacaagacccacacctgtcccccttgtcctgcccctgaactgctgggcggacctagcgtgttcctgttccccccaaagcccaaggataccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtctgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccactccccccgtgctggactccgacggctcattcttcctgtacagcaagctgacagtggacaagagccggtggcagcagggcaacgtgttcagctgcagcgtgatgcacgaggccctgcacaaccactacacccagaagtccctgagcctgagccctggc

SEQ ID NO: 27 - DNA, 1539 bp, Artificial Sequence
  polynucleotide encoding PDGFRbeta-D1-D3-EPKSC-hIgG1-Fc
ctggtcgtgacacctcctggcccagaactggtgctgaacgtgtccagcaccttcgtgctgacctgcagcggatctgcccctgtcgtgtgggagcggatgagccaggaaccccctcaggaaatggccaaggcccaggacggcaccttcagcagcgtgctgacactgaccaacctgaccggcctggataccggcgagtacttctgcacccacaacgacagcagaggcctggaaaccgatgagcggaagcggctgtacatcttcgtgcccgatcctaccgtgggcttcctgcccaacgatgccgaggaactgttcatcttcctgaccgagattaccgagatcaccatcccctgcagagtgaccgacccccagctggtcgtgaccctgcacgagaagaaaggcgacgtggccctgcccgtgccctacgatcatcagagaggcttcagcggcatcttcgaggacagaagctacatctgcaagaccaccatcggcgacagagaggtggacagcgacgcctactacgtgtaccggctgcaggtgtcctccatcaacgtgtccgtgaacgccgtgcagaccgtcgtgcggcagggcgagaatatcaccctgatgtgcatcgtgatcggcaacgaggtcgtgaacttcgagtggacctacccccggaaagaaagcggcagactggtggaacccgtgaccgacttcctgctggacatgccctaccacatccggtccatcctgcacatccctagcgccgagctggaagatagcggcacctacacctgtaacgtgaccgagagcgtgaacgaccaccaggacgagaaggccatcaacatcaccgtggtggaaagcggcgagcccaagagctgcgacaagacccacacctgtcccccttgtcctgcccctgaactgctgggcggacctagcgtgttcctgttccccccaaagcccaaggataccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtctgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccactccccccgtgctggactccgacggctcattcttcctgtacagcaagctgacagtggacaagagccggtggcagcagggcaacgtgttcagctgcagcgtgatgcacgaggccctgcacaaccactacacccagaagtccctgagcctgagccctggc

SEQ ID NO: 28 - DNA, 1539 bp, Artificial Sequence
  polynucleotide encoding PDGFRbeta-D1-D3-EPKSS-hIgG1-Fc
ctggtcgtgacacctcctggcccagaactggtgctgaacgtgtccagcaccttcgtgctgacctgcagcggatctgcccctgtcgtgtgggagcggatgagccaggaaccccctcaggaaatggccaaggcccaggacggcaccttcagcagcgtgctgacactgaccaacctgaccggcctggataccggcgagtacttctgcacccacaacgacagcagaggcctggaaaccgatgagcggaagcggctgtacatcttcgtgcccgatcctaccgtgggcttcctgcccaacgatgccgaggaactgttcatcttcctgaccgagattaccgagatcaccatcccctgcagagtgaccgacccccagctggtcgtgaccctgcacgagaagaaaggcgacgtggccctgcccgtgccctacgatcatcagagaggcttcagcggcatcttcgaggacagaagctacatctgcaagaccaccatcggcgacagagaggtggacagcgacgcctactacgtgtaccggctgcaggtgtcctccatcaacgtgtccgtgaacgccgtgcagaccgtcgtgcggcagggcgagaatatcaccctgatgtgcatcgtgatcggcaacgaggtcgtgaacttcgagtggacctacccccggaaagaaagcggcagactggtggaacccgtgaccgacttcctgctggacatgccctaccacatccggtccatcctgcacatccctagcgccgagctggaagatagcggcacctacacctgtaacgtgaccgagagcgtgaacgaccaccaggacgagaaggccatcaacatcaccgtggtggaaagcggcgagcccaagagcagcgacaagacccacacctgtcccccttgtcctgcccctgaactgctgggcggacctagcgtgttcctgttccccccaaagcccaaggataccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtctgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccactccccccgtgctggactccgacggctcattcttcctgtacagcaagctgacagtggacaagagccggtggcagcagggcaacgtgttcagctgcagcgtgatgcacgaggccctgcacaaccactacacccagaagtccctgagcctgagccctggc

SEQ ID NO: 29 - DNA, 1536 bp, Artificial Sequence
  polynucleotide encoding PDGFRbeta-D1-D3-GGGGG-hIgG1-Fc
ctggtcgtgacacctcctggcccagaactggtgctgaacgtgtccagcaccttcgtgctgacctgcagcggatctgcccctgtcgtgtgggagcggatgagccaggaaccccctcaggaaatggccaaggcccaggacggcaccttcagcagcgtgctgacactgaccaacctgaccggcctggataccggcgagtacttctgcacccacaacgacagcagaggcctggaaaccgatgagcggaagcggctgtacatcttcgtgcccgatcctaccgtgggcttcctgcccaacgatgccgaggaactgttcatcttcctgaccgagattaccgagatcaccatcccctgcagagtgaccgacccccagctggtcgtgaccctgcacgagaagaaaggcgacgtggccctgcccgtgccctacgatcatcagagaggcttcagcggcatcttcgaggacagaagctacatctgcaagaccaccatcggcgacagagaggtggacagcgacgcctactacgtgtaccggctgcaggtgtcctccatcaacgtgtccgtgaacgccgtgcagaccgtcgtgcggcagggcgagaatatcaccctgatgtgcatcgtgatcggcaacgaggtcgtgaacttcgagtggacctacccccggaaagaaagcggcagactggtggaacccgtgaccgacttcctgctggacatgccctaccacatccggtccatcctgcacatccctagcgccgagctggaagatagcggcacctacacctgtaacgtgaccgagagcgtgaacgaccaccaggacgagaaggccatcaacatcaccgtggtggaaagcggcggcggaggcggagacaagacccacacctgtcccccttgtcctgcccctgaactgctgggcggacctagcgtgttcctgttccccccaaagcccaaggataccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtctgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccactccccccgtgctggactccgacggctcattcttcctgtacagcaagctgacagtggacaagagccggtggcagcagggcaacgtgttcagctgcagcgtgatgcacgaggccctgcacaaccactacacccagaagtccctgagcctgagccctggc

SEQ ID NO: 30 - DNA, 1539 bp, Artificial Sequence
  polynucleotide encoding PDGFRbeta-D1-D3-1xGGGGS-hIgG1-Fc
ctggtcgtgacacctcctggcccagaactggtgctgaacgtgtccagcaccttcgtgctgacctgcagcggatctgcccctgtcgtgtgggagcggatgagccaggaaccccctcaggaaatggccaaggcccaggacggcaccttcagcagcgtgctgacactgaccaacctgaccggcctggataccggcgagtacttctgcacccacaacgacagcagaggcctggaaaccgatgagcggaagcggctgtacatcttcgtgcccgatcctaccgtgggcttcctgcccaacgatgccgaggaactgttcatcttcctgaccgagattaccgagatcaccatcccctgcagagtgaccgacccccagctggtcgtgaccctgcacgagaagaaaggcgacgtggccctgcccgtgccctacgatcatcagagaggcttcagcggcatcttcgaggacagaagctacatctgcaagaccaccatcggcgacagagaggtggacagcgacgcctactacgtgtaccggctgcaggtgtcctccatcaacgtgtccgtgaacgccgtgcagaccgtcgtgcggcagggcgagaatatcaccctgatgtgcatcgtgatcggcaacgaggtcgtgaacttcgagtggacctacccccggaaagaaagcggcagactggtggaacccgtgaccgacttcctgctggacatgccctaccacatccggtccatcctgcacatccctagcgccgagctggaagatagcggcacctacacctgtaacgtgaccgagagcgtgaacgaccaccaggacgagaaggccatcaacatcaccgtggtggaaagcggcggcggaggcggatccgacaagacccacacctgtcccccttgtcctgcccctgaactgctgggcggacctagcgtgttcctgttccccccaaagcccaaggataccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtctgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccactccccccgtgctggactccgacggctcattcttcctgtacagcaagctgacagtggacaagagccggtggcagcagggcaacgtgttcagctgcagcgtgatgcacgaggccctgcacaaccactacacccagaagtccctgagcctgagccctggc

SEQ ID NO: 31 - DNA, 1554 bp, Artificial Sequence
  polynucleotide encoding PDGFRbeta-D1-D3-2xGGGGS-hIgG1-Fc
ctggtcgtgacacctcctggcccagaactggtgctgaacgtgtccagcaccttcgtgctgacctgcagcggatctgcccctgtcgtgtgggagcggatgagccaggaaccccctcaggaaatggccaaggcccaggacggcaccttcagcagcgtgctgacactgaccaacctgaccggcctggataccggcgagtacttctgcacccacaacgacagcagaggcctggaaaccgatgagcggaagcggctgtacatcttcgtgcccgatcctaccgtgggcttcctgcccaacgatgccgaggaactgttcatcttcctgaccgagattaccgagatcaccatcccctgcagagtgaccgacccccagctggtcgtgaccctgcacgagaagaaaggcgacgtggccctgcccgtgccctacgatcatcagagaggcttcagcggcatcttcgaggacagaagctacatctgcaagaccaccatcggcgacagagaggtggacagcgacgcctactacgtgtaccggctgcaggtgtcctccatcaacgtgtccgtgaacgccgtgcagaccgtcgtgcggcagggcgagaatatcaccctgatgtgcatcgtgatcggcaacgaggtcgtgaacttcgagtggacctacccccggaaagaaagcggcagactggtggaacccgtgaccgacttcctgctggacatgccctaccacatccggtccatcctgcacatccctagcgccgagctggaagatagcggcacctacacctgtaacgtgaccgagagcgtgaacgaccaccaggacgagaaggccatcaacatcaccgtggtggaaagcggcggcggaggcggatccggcggcggaggaagtgacaagacccacacctgtcccccttgtcctgcccctgaactgctgggcggacctagcgtgttcctgttccccccaaagcccaaggataccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtctgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccactccccccgtgctggactccgacggctcattcttcctgtacagcaagctgacagtggacaagagccggtggcagcagggcaacgtgttcagctgcagcgtgatgcacgaggccctgcacaaccactacacccagaagtccctgagcctgagccctggc

SEQ ID NO: 32 - DNA, 1569 bp, Artificial Sequence
  polynucleotide encoding PDGFRbeta-D1-D3-3xGGGGS-hIgG1-Fc
ctggtggtgacacctcccggccctgagctggtgctgaacgtgtcctccaccttcgtgctgacctgctccggctccgcccctgtggtctgggagcggatgtcccaggaacccccccaggaaatggccaaggcccaggacggcaccttctcctccgtgctgaccctgaccaacctgaccggcctggataccggcgagtacttctgcacccacaacgactctcggggcctggaaaccgacgagcggaagcggctgtacatcttcgtgcccgaccctaccgtgggcttcctgcccaacgacgccgaggaactgttcatcttcctgaccgagattaccgagatcaccatcccctgcagagtgaccgacccccagctggtcgtgaccctgcacgagaagaaaggcgacgtggccctgcccgtgccctacgaccaccagagaggcttctccggcatcttcgaggaccggtcttacatctgcaagaccaccatcggcgacagagaggtggactccgacgcctactacgtgtaccggctgcaggtgtcctccatcaacgtgtccgtgaacgccgtgcagaccgtggtccgacagggcgagaacatcaccctgatgtgcatcgtgatcggcaacgaggtggtgaacttcgagtggacctacccccggaaagaatctggccggctggtggaacccgtgaccgacttcctgctggacatgccctaccacatccggtccatcctgcacatcccttccgccgagctggaagattccggcacctacacctgtaacgtgaccgagagcgtgaacgatcaccaggacgagaaggccatcaacatcaccgtggtggaatctggcggcggaggcggaagtggtggagggggatctgggggaggcggctctgataagacccacacctgcccaccctgccctgcccctgaactgctgggcggaccctccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtctgtgctgaccgtgctgcaccaggactggctgaacggcaaagagtacaagtgcaaggtctccaacaaggccctgcctgcccccatcgaaaagaccatctccaaggccaagggccagccccgcgagccccaggtgtacaccctgccccctagccgggacgagctgaccaagaaccaggtgtccctgacctgcctggtgaaaggcttctacccctccgatatcgccgtggaatgggagtccaacggccagcccgagaacaactacaagaccaccccccctgtgctggacagcgacggctcattcttcctgtactccaagctgaccgtggacaagtcccggtggcagcagggcaacgtgttctcctgctccgtgatgcacgaggccctgcacaaccactacacccagaagtccctgtccctgagccccggc

SEQ ID NO: 33 - DNA, 1584 bp, Artificial Sequence
  polynucleotide encoding PDGFRbeta-D1-D3-4xGGGGS-hIgG1-Fc
ctggtcgtgacacctcctggcccagaactggtgctgaacgtgtccagcaccttcgtgctgacctgcagcggatctgcccctgtcgtgtgggagcggatgagccaggaaccccctcaggaaatggccaaggcccaggacggcaccttcagcagcgtgctgacactgaccaacctgaccggcctggataccggcgagtacttctgcacccacaacgacagcagaggcctggaaaccgatgagcggaagcggctgtacatcttcgtgcccgatcctaccgtgggcttcctgcccaacgatgccgaggaactgttcatcttcctgaccgagattaccgagatcaccatcccctgcagagtgaccgacccccagctggtcgtgaccctgcacgagaagaaaggcgacgtggccctgcccgtgccctacgatcatcagagaggcttcagcggcatcttcgaggacagaagctacatctgcaagaccaccatcggcgacagagaggtggacagcgacgcctactacgtgtaccggctgcaggtgtcctccatcaacgtgtccgtgaacgccgtgcagaccgtcgtgcggcagggcgagaatatcaccctgatgtgcatcgtgatcggcaacgaggtcgtgaacttcgagtggacctacccccggaaagaaagcggcagactggtggaacccgtgaccgacttcctgctggacatgccctaccacatccggtccatcctgcacatccctagcgccgagctggaagatagcggcacctacacctgtaacgtgaccgagagcgtgaacgaccaccaggacgagaaggccatcaacatcaccgtggtggaaagcggcggcggaggcggatccggcggcggaggaagtggcgggggaggatctggaggaggaggctctgacaagacccacacctgtcccccttgtcctgcccctgaactgctgggcggacctagcgtgttcctgttccccccaaagcccaaggataccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtctgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccactccccccgtgctggactccgacggctcattcttcctgtacagcaagctgacagtggacaagagccggtggcagcagggcaacgtgttcagctgcagcgtgatgcacgaggccctgcacaaccactacacccagaagtccctgagcctgagccctggc

SEQ ID NO: 34 - DNA, 1599 bp, Artificial Sequence
  polynucleotide encoding PDGFRbeta-D1-D3-5xGGGGS-hIgG1-Fc
ctggtcgtgacacctcctggcccagaactggtgctgaacgtgtccagcaccttcgtgctgacctgcagcggatctgcccctgtcgtgtgggagcggatgagccaggaaccccctcaggaaatggccaaggcccaggacggcaccttcagcagcgtgctgacactgaccaacctgaccggcctggataccggcgagtacttctgcacccacaacgacagcagaggcctggaaaccgatgagcggaagcggctgtacatcttcgtgcccgatcctaccgtgggcttcctgcccaacgatgccgaggaactgttcatcttcctgaccgagattaccgagatcaccatcccctgcagagtgaccgacccccagctggtcgtgaccctgcacgagaagaaaggcgacgtggccctgcccgtgccctacgatcatcagagaggcttcagcggcatcttcgaggacagaagctacatctgcaagaccaccatcggcgacagagaggtggacagcgacgcctactacgtgtaccggctgcaggtgtcctccatcaacgtgtccgtgaacgccgtgcagaccgtcgtgcggcagggcgagaatatcaccctgatgtgcatcgtgatcggcaacgaggtcgtgaacttcgagtggacctacccccggaaagaaagcggcagactggtggaacccgtgaccgacttcctgctggacatgccctaccacatccggtccatcctgcacatccctagcgccgagctggaagatagcggcacctacacctgtaacgtgaccgagagcgtgaacgaccaccaggacgagaaggccatcaacatcaccgtggtggaaagcggcggcggaggcggatccggcggcggaggaagtggcgggggaggatctggaggaggaggctctggcggcggaggctctgacaagacccacacctgtcccccttgtcctgcccctgaactgctgggcggacctagcgtgttcctgttccccccaaagcccaaggataccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtctgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccactccccccgtgctggactccgacggctcattcttcctgtacagcaagctgacagtggacaagagccggtggcagcagggcaacgtgttcagctgcagcgtgatgcacgaggccctgcacaaccactacacccagaagtccctgagcctgagccctggc

SEQ ID NO: 35 - DNA, 1614 bp, Artificial Sequence
  polynucleotide encoding PDGFRbeta-D1-D3-6xGGGGS-hIgG1-Fc
ctggtcgtgacacctcctggcccagaactggtgctgaacgtgtccagcaccttcgtgctgacctgcagcggatctgcccctgtcgtgtgggagcggatgagccaggaaccccctcaggaaatggccaaggcccaggacggcaccttcagcagcgtgctgacactgaccaacctgaccggcctggataccggcgagtacttctgcacccacaacgacagcagaggcctggaaaccgatgagcggaagcggctgtacatcttcgtgcccgatcctaccgtgggcttcctgcccaacgatgccgaggaactgttcatcttcctgaccgagattaccgagatcaccatcccctgcagagtgaccgacccccagctggtcgtgaccctgcacgagaagaaaggcgacgtggccctgcccgtgccctacgatcatcagagaggcttcagcggcatcttcgaggacagaagctacatctgcaagaccaccatcggcgacagagaggtggacagcgacgcctactacgtgtaccggctgcaggtgtcctccatcaacgtgtccgtgaacgccgtgcagaccgtcgtgcggcagggcgagaatatcaccctgatgtgcatcgtgatcggcaacgaggtcgtgaacttcgagtggacctacccccggaaagaaagcggcagactggtggaacccgtgaccgacttcctgctggacatgccctaccacatccggtccatcctgcacatccctagcgccgagctggaagatagcggcacctacacctgtaacgtgaccgagagcgtgaacgaccaccaggacgagaaggccatcaacatcaccgtggtggaaagcggcggcggaggcggatccggcggcggaggaagtggcgggggaggatctggaggaggaggctctggcggcggaggctctggcggcggaggaagtgacaagacccacacctgtcccccttgtcctgcccctgaactgctgggcggacctagcgtgttcctgttccccccaaagcccaaggataccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtctgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccactccccccgtgctggactccgacggctcattcttcctgtacagcaagctgacagtggacaagagccggtggcagcagggcaacgtgttcagctgcagcgtgatgcacgaggccctgcacaaccactacacccagaagtccctgagcctgagccctggc

SEQ ID NO: 36 - DNA, 1629 bp, Artificial Sequence
  polynucleotide encoding PDGFRbeta-D1-D3-7xGGGGS-hIgG1-Fc
ctggtcgtgacacctcctggcccagaactggtgctgaacgtgtccagcaccttcgtgctgacctgcagcggatctgcccctgtcgtgtgggagcggatgagccaggaaccccctcaggaaatggccaaggcccaggacggcaccttcagcagcgtgctgacactgaccaacctgaccggcctggataccggcgagtacttctgcacccacaacgacagcagaggcctggaaaccgatgagcggaagcggctgtacatcttcgtgcccgatcctaccgtgggcttcctgcccaacgatgccgaggaactgttcatcttcctgaccgagattaccgagatcaccatcccctgcagagtgaccgacccccagctggtcgtgaccctgcacgagaagaaaggcgacgtggccctgcccgtgccctacgatcatcagagaggcttcagcggcatcttcgaggacagaagctacatctgcaagaccaccatcggcgacagagaggtggacagcgacgcctactacgtgtaccggctgcaggtgtcctccatcaacgtgtccgtgaacgccgtgcagaccgtcgtgcggcagggcgagaatatcaccctgatgtgcatcgtgatcggcaacgaggtcgtgaacttcgagtggacctacccccggaaagaaagcggcagactggtggaacccgtgaccgacttcctgctggacatgccctaccacatccggtccatcctgcacatccctagcgccgagctggaagatagcggcacctacacctgtaacgtgaccgagagcgtgaacgaccaccaggacgagaaggccatcaacatcaccgtggtggaaagcggcggcggaggcggatccggcggcggaggaagtggcgggggaggatctggaggaggaggctctggcggcggaggctctggcggcggaggaagtggcggagggggctctgacaagacccacacctgtcccccttgtcctgcccctgaactgctgggcggacctagcgtgttcctgttccccccaaagcccaaggataccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtctgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccactccccccgtgctggactccgacggctcattcttcctgtacagcaagctgacagtggacaagagccggtggcagcagggcaacgtgttcagctgcagcgtgatgcacgaggccctgcacaaccactacacccagaagtccctgagcctgagccctggc

(19) United States
(12) Patent Application Publication
(10) Pub. No.: US 2016/0009776 A1
(43) Pub. Date: Jan. 14, 2016

(54) PDGFRBETA-FC FUSION PROTEINS AND USES THEREOF

(71) Applicant: Bayer Pharma Aktiengesellschaft, Berlin (DE)

(72) Inventors: Lars Linden, Gevelsberg (DE); Thomas Schlange, Haan (DE); Andreas Wilmen, Koln (DE); Mark Trautwein, Wulfrath (DE); Tibor Schomber, Wulfrath (DE); Michael Bottger, Wuppertal (DE); Jürgen Klar, Wuppertal (DE); Simone Greven, Dormagen (DE)

(21) Appl. No.: 14/328,487

(22) Filed: Jul. 10, 2014

(51) Int. Cl. C07K 14/49

(52) U.S. Cl. CPC C07K 14/49; C07K 2319/30

(57) ABSTRACT

The present disclosure provides PDGFRbeta-Fc fusion proteins, or biologically active fragments thereof, comprising an extracellular domain of PDGFRbeta, or a biologically active fragment thereof, a linker and a Fc domain, wherein said fusion protein binds one or more of platelet-derived growth factor ligands with high affinity. The PDGFRbeta-Fc fusion proteins, or biologically active fragments thereof, accordingly, can be used to treat pathological neovascularization and fibrosis, e.g. cancer, ocular neovascular disorders or nephropathies. The disclosure also provides methods for treating ocular neovascular disorders with these fusion proteins without increasing vascular leakage. Such PDGFRbeta-Fc fusion proteins, or biologically active fragments thereof, exhibit increased terminal half time in the eye. Also provided are nucleic acid sequences encoding the foregoing PDGFRbeta-Fc fusion proteins, or biologically active fragments thereof, vectors containing the same, pharmaceutical compositions and kits with instructions for use.

### SEQUENCE LISTING

The instant application contains a Sequence Listing which has been submitted via EFS-Web and is hereby incorporated by reference in its entirety. Said ASCII copy, created on Jul. 10, 2014, is named BCPP_{—}236_{—}101_Sequence_Listing_PTO.txt, and is 95,346 bytes in size.

### BACKGROUND OF THE DISCLOSURE

The Platelet-Derived Growth Factor (PDGF) family consists of four members (A-D) that share a cluster of 8 cysteine residues. PDGFs form biologically active homodimers and one heterodimer (PDGF-AB) via disulfide bonds. They bind to two closely related receptor tyrosine kinases, PDGFRalpha and -beta, which homo- or heterodimerize upon ligand binding. The specificity of the different PDGF ligands for both receptors varies, with PDGF-BB and -DD binding PDGFRbeta, and PDGF-BB, PDGF-CC and -AA binding PDGFRalpha. PDGF-BB, PDGF-AB and to a weaker extent PDGF-CC and -DD were also reported to bind and activate the heterodimeric PDGFRalpha/beta (Heldin and Westermark, Physiol Rev 1999; 79(4): 1283-1316; Reigstad et al., FEBS J 2005; 272: 5723-5741).

Downstream of PDGFR activation p85 and Grb2 scaffolding proteins, among others, are recruited to the phosphorylated receptor and initiate the activation of the MAPK and PI3K/AKT signaling pathways. Thereby, PDGF signaling regulates proliferation, survival and migration of PDGFRalpha or -beta expressing cells. Both receptors are found on vascular smooth muscle cells and pericytes where PDGFRbeta stimulates migration, while PDGFRalpha appears to have an anti-migratory function (Yokote et al., J Biol Chem 1996; 271: 5101-5111). Anti-apoptotic and pro-proliferative functions have been attributed to both receptors (Yao and Cooper, Science 1995; 267: 2003-2006).

In the context of vascular biology, PDGFs play an important role in maintaining vessel integrity (for comprehensive recent review cf. Goel et al., Physiol Rev 2011; 91: 1071-1121). Endothelial cells in newly formed vessels secrete PDGFs to attract pericytes to their walls. This process is important in vessel maturation and leads to varying degrees of pericyte coverage on the mature vessels. Importantly, endothelial cells in mature vessels become largely independent of VEGF signaling for survival. Only when an external stimulus like injury, inflammation or hypoxia triggers neo-angiogenesis, is responsiveness to VEGF restored. When PDGF signaling is blocked by pharmacological means or is deregulated in disease, e.g. during tumor angiogenesis, vessels become or remain leaky. As a consequence, fluid and soluble plasma components drain into surrounding tissue, leaving more distant areas in shortage of oxygen and nutrients. This has been demonstrated in different disease models using systemic administration of a PDGF-B aptamer for models of lung inflammation (Fuxe et al., Am J Pathol 2011; 178 (6):2897-2909) and tumor angiogenesis in the transgenic RIP-Tag2 model (Falcon et al., Am J Pathol 2011; 178 (6):2920-2930). The redundancy of the PDGF ligands in regulating vascular leakage has also been shown in transgenic mice overexpressing PDGF-D together with VEGF-E in the skin where PDGF-DD is increasing pericyte coverage of newly formed vessels and reduces vascular leakage (Uutela et al., Blood 2004; 104: 3198-3204).

Neovascular disease of the retina causes loss of vision and blindness via the formation of new vessels in the retina and increased vascular permeability resulting in leakage of plasma proteins into the vitreous of patients. Therapeutic approaches to limit the formation of new vessels in the retina are so far not successful in all patients. In fact, in more than 50% of the reported cases visual acuity does not improve or declines again after initial improvement. Restoring vision in these patients to an extent that enables patients to read or drive is therefore an important aim of research in the field.

A role for PDGF signaling in retinal and choroidal neovascularization was shown in both preclinical models and in the clinical setting. In a rat model of Retinopathy of Prematurity (ROP) systemic treatment with the PDGFR inhibitor STI571 induced apoptosis in pericytes of the inner retina (Wilkinson-Berka et al., Am J Pathol 2004; 164(4): 1263-1273). Preclinical data imply that not only PDGF-DD (Kumar et al., J Biol Chem 2010; 285(20):15500-15510) and PDGF-BB (Akiyama et al., J Cell Physiol 2006; 207:407-412) as ligands for PDGFRbeta are involved in choroidal neovascularization and proliferative vitreoretinopathy, but also the PDGFRalpha ligand PDGF-CC. An antibody targeting the latter reduced lesion size in the mouse laser CNV model (Hou et al., PNAS 2010; 107(27):12216-12221). This underlines the need for a therapeutic approach that addresses the redundancy in PDGF signaling for the treatment of neovascular disease of the retina.

A combination of PDGFRbeta antibody with a VEGF-A aptamer resulted in additive reduction of neovascularization in a mouse laser CNV model both in a preventive and therapeutic setting (Jo et al., Am J Pathol 2006; 168(6):2036-2053). Importantly, the clinical viability of targeting PDGF-BB in wet age-related macular degeneration (wAMD) was supported by the results of a phase Ilb trial with the PDGF-B aptamer Fovista in combination with Lucentis® (ranibizumab injection) demonstrating superior efficacy over Lucentis monotherapy in patients with wAMD (http://www.sec.gov/Archives/edgar/data/1410939/000119312513336689/d560505ds1.htm).

A downside of targeting PDGF signaling in angiogenesis has been that several studies in preclinical models demonstrated that reducing the pericyte coverage of blood vessels increases vascular leakage, i.e. the leakage of serum protein into the surrounding tissues. Such increase has been observed using aptamers binding to PDGF-B, anti-PDGFRbeta function-blocking antibodies and the PDGFR tyrosine kinase inhibitor imatinib (Ruan J., et al., Blood 121: 5192-5202, Epub Apr. 30 2013; Uemura, A., et al., J Clin Invest 110:1619-1628, December 2002; Fuxe et al., Am J Pathol 178 (6):2897-2909, Epub May 6th 2011; Falcon et al., Am J Pathol 178 (6):2920-2930, June 2011). Increased vascular leakage, however, is considered an adverse effect in the treatment of neovascular diseases of the eye in humans, as it causes vision loss by blurring the vitreous humor (Horster, R. et al., Graefes Arch Clin Exp Ophthalmol 2011 249:645-652, Epub Dec. 18 2010; Kent, D. L. Molecular Vision 20:46-55, Jan. 6 2014). This is of particular importance in diabetic retinopathy, where a loss of pericytes is part of the pathological degeneration of retinal vessels (Klaassen, I. et al., Progress in Retinal and Eye Research 34: 19e48, Feb. 13 2013). It is therefore desirable to combine the anti-angiogenic properties of an anti-PDGF therapeutic approach with preservation of vessel function and integrity.

The blockade of PDGF signaling using a fusion protein consisting of the extracellular portion of PDGFRbeta or fragments thereof like the ligand binding domains D1-D3 and the Fc-part of an antibody has been described previously (Duan et al., J Biol Chem 266(1): 413-418, Jan. 5 1991; Heidaran, M. A. et al., FASEBJ 9: 140-145, January 1995; Wolf, D. et al., U.S. Pat. No. 5,686,572). The use of PDGFR beta Fc decoy receptors in vivo without a peptide linker inserted between receptor and Fc portion has been reported to reduce tumor microvascular density in a triple-negative breast cancer xenograft model (Shan et al., Cancer Sci 102 (10): 1904-1910, October 2011) but showed no impact on vessel density in ovarian xenograft models (Lu et al., Am J Obstet Gynecol 198: 477.e1-477.e10, April 2008). None of the published reports on PDGF decoy receptors gives a hint to the effect on vascular integrity and permeability in the eye or in any other tissue or compartment.

Full IgG antibodies have an exceptionally long half-life, while small antibody derivatives or other biologics formats often suffer from rapid elimination from circulation. In order to improve administration and therapeutic efficacy, modifications to extend the plasma half-life have been developed and implemented in these biologics formats. Methods for half-life extension include PEGylation, HESylation, glycosylation and polysialation. Another approach to achieve longer half-life is fusion of target proteins to an immunoglobin Fc domain or to human serum albumin. Also described are fusions to albumin binding domains of bacterial origin, to albumin binding peptides or albumin binding antibodies or Fc-binding moieties (Kontermann, Biodrugs 23(2):93-109, 2009; Kontermann, Therapeutic Proteins, Strategies to Modulate their Plasma Half-lives, Wiley, 2010 ISBN:978-3-527-32849-9; Sleep et al. Biochim. Biophys. Acta 1830:5526-5534, 20133). However, little is known about the half-life extension of proteins in the retina. Potential contributions of neonatal Fc (FcRn) immunoglobulin (IgG) receptor/transporter and choroidal neovascularization in vitreal clearance has been discussed in Kim et al. (Molecular Vision, 15:2803-2812, 2009).

Despite the progress described in the art, there remains a need for improved medicines for the treatment of pathological neovascularization and fibrosis especially for the treatment of ocular neovascular disorders like AMD. In particular there remains a need for an anti-PDGF therapeutic with increased binding affinity, increased potency and extended vitreal half-life which reduces neovascularization but does not increase vascular leakage in contrast to other known anti-PDGF therapeutics. Additionally, there is a need for an anti-PDGF therapeutic which not only does not increase vascular leakage but even reduces vascular leakage, a known adverse effect in the treatment of neovascular diseases of the eye, when used in a combination therapy with a second receptor tyrosine kinase signaling antagonist e.g. an anti-VEGF therapeutic.

The PDGFRbeta-Fc fusion proteins according to the invention which comprise a linker between the PDGFRbeta portion and the Fc portion fulfill these needs. Usually linkers are included in fusion proteins to enhance expression, to reduce neoantigenicity or to allow proper folding of the individual moieties which may increase the binding activity by permitting more freedom to the active moiety (no steric hindrance). The PDGFRbeta-Fc fusion proteins according to the invention also show a modest increase in binding affinity compared to a linker-less PDGFRbeta fusion protein, but in this case it is accompanied by an unexpected many times higher increase in potency. Furthermore, the linker-containing PDGFRbeta-Fc fusion proteins according to the invention show a significant and unexpected increase in intravitreal terminal half-life compared to the linker-less variant. Additionally, PDGFRbeta-Fc fusion proteins show the ability to reduce neovascularization without increasing, and even decreasing, vascular leakage, either as a stand-alone therapy or a combination therapy together with a receptor tyrosine kinase signaling antagonist, which is in stark contrast to other known anti-PDGF therapeutics. In addition, the linker-containing PDGFRbeta-Fc fusion proteins according to the invention show superior properties both in reducing neovascularization and concerning vascular leakage, when compared to the linker-less variant or compared with other PDGF-directed therapeutics.

### SUMMARY OF THE DISCLOSURE

The disclosure relates to PDGFRbeta-Fc fusion proteins, or biologically active fragments thereof, that exhibit increased in vitro potency and extended vitreal half-life in vivo, which, in some embodiments, is useful for the treatment of neovascular or fibrotic disease especially of the eye, in particular of the retina, e.g., but not limited to, wet age-related macular degeneration and posterior vitreoretinopathy, either alone or in combination with other anti-angiogenic regimens such as, but not limited to, one or more of anti-VEGF, anti-VEGFR, anti-HGF, anti-HGFR, anti-FGF, anti-FGFR anti-IGF, or anti-IFGR agents.

In one aspect, the disclosure provides a PDGFRbeta-Fc fusion protein comprising an extracellular domain of PDGFRbeta and a Fc domain, wherein the PDGFRbeta-Fc fusion protein binds one or more of PDGF ligands -BB, -DD, and -AB with a K_{D} of less than 300 nM, less than 250 nM, less than 200 nM, less than 150 nM, less than 100 nM, less than 50 nM, less than 10 nM, less than 1 nM, less than 500 pM, less than 200 pM, less than 125 pM, less than 100 pM, less than 50 pM, less than 30 pM or less than 10 pM. In some embodiments, the PDGFRbeta-Fc fusion protein binds one or more of PDGF ligands -BB, -DD, and -AB with a K_{D} of between 1-100 pM, between 100-500 pM, between 500-1000 pM, between 1 nM and 100 nM, or between 100-500 nM. In some embodiments, the PDGFRbeta-Fc fusion protein does not substantially bind to platelet-derived growth factor ligands AA and CC. For example, the PDGFRbeta-Fc fusion protein may bind to PDGF ligands AA or CC with no meaningful binding affinity, such as it may bind to the ligands AA and CC with a K_{D} of greater than 300 nM, greater than 400 nM, greater than 500 nM, or greater than 1 μM. In some embodiments, the PDGFRbeta extracellular domain of the fusion protein binds one or more of the PDGF ligands -BB and -DD with a K_{D} of less than 200 pM, or less than 125 pM, or less than 100 pM, or less than 50 pM, or less than 30 pM. In some embodiments, the PDGFRbeta extracellular domain of the fusion protein binds one or more of the PDGF ligands -BB and -DD with a K_{D} of 1 to 500 pM, 1 to 200 pM, 1 to 100 pM, 1 to 50 pM, 50 to 150 pM or 100 to 200 pM. The fusion protein may further comprise a polypeptide linker domain connecting the PDGFRbeta extracellular domain and the Fc domain. In some embodiments, the linker has a length of at least 12, 15, 20, 25, 30 or 35 amino acids. In other embodiments, the linker has the amino acid sequence (GGGGS)n, where n is 3. The linker may also have the amino acid sequence (GGGGS)n where n is 3-10. Alternatively, n may be 3-7. In some embodiments, n is 3, 4, 5, 6, 7, 8, 9 or 10. In other embodiments, n is 4.

In another aspect, the disclosure provides a PDGFRbeta-Fc fusion protein comprising an extracellular domain of PDGFRbeta and a Fc domain, wherein the extracellular domain of PDGFRbeta binds a PDGF ligand BB with a K_{D} of less than 200 pM, or less than 125 pM, or less than 100 pM, or less than 50 pM, or less than 30 pM. In some embodiments, the PDGFRbeta extracellular domain of the fusion protein binds the PDGF ligand -BB with a K_{D} of 1 to 500 pM, 1 to 200 pM, 1 to 100 pM, 1 to 50 pM, 50 to 150 pM or 100 to 200 pM. The fusion protein may further comprise a polypeptide linker domain connecting the PDGFRbeta extracellular domain and the Fc domain. In some embodiments, the linker has a length of at least 12, 15, 20, 25, 30, 35, 40, 45, or 50 amino acids. In other embodiments, the linker has the amino acid sequence (GGGGS)n, where n is 3. The linker may also have the amino acid sequence (GGGGS)n where n is 3-10. Alternatively, n may be 3-7. In some embodiments, n is 3, 4, 5, 6, 7, 8, 9 or 10. In other embodiments, n is 4.

In another aspect, the disclosure provides a PDGFRbeta-Fc fusion protein comprising an extracellular domain of PDGFRbeta and a Fc domain, wherein the extracellular domain of PDGFRbeta binds a PDGF ligand DD with a K_{D} of less than 200 pM, or less than 125 pM, or less than 100 pM, or less than 50 pM, or less than 30 pM. In some embodiments, the PDGFRbeta extracellular domain of the fusion protein binds the PDGF ligand -DD with a K_{D} of 1 to 500 pM, 1 to 200 pM, 1 to 100 pM, 1 to 50 pM, 50 to 150 pM or 100 to 200 pM.

In some embodiments, the PDGFRbeta portion is connected to the Fc portion by means of a linker. In some embodiments, the linkers are glycine and serine rich linkers. Other near neutral amino acids, such as, but not limited to, Thr, Asn, Pro and Ala, may also be used in the linker sequence. In some embodiments, the linker comprises various permutations of amino acid sequences containing Gly and Ser. In some embodiments, the linkers have a length of at least 12, 15, 20, 25, 30, 35, 40, 45 or 50 amino acids. In preferred embodiments, the linker comprises the amino acid sequence GlyGlyGlyGlySer (GGGGS), or repetitions thereof (GGGGS)n, where n 1. In more preferred embodiments n 3, or n=3-10. In even more preferred embodiments n=3-7, or n=3, 4, 5, 6, or 7. In further preferred embodiments, n=4.

In certain embodiments of any of the foregoing or following, the extracellular domain of PDGFRbeta comprises an amino acid sequence that is at least 90%, 95%, 96%, 97%, 98%, 99% or 100% identical to SEQ ID NO: 2. In other embodiments of any of the foregoing or following, the extracellular domain of PDGFRbeta comprises an amino acid sequence that is at least 90%, 95%, 96%, 97%, 98%, 99% or 100% identical to SEQ ID NO: 3.

In certain embodiments of any of the foregoing or following embodiments, the Fc domain comprises a mammalian Fc domain. In other embodiments, Fc domain comprises a human Fc domain. In some embodiments, the Fc domain is derived from an immunoglobin selected from IgG1, IgG2, IgG3, or IgG4. In other embodiments, the Fc domain is derived from a human IgG1. In yet another embodiment, the Fc domain comprises a sequence that is at least 90%, 95%, 96%, 97%, 98%, 99% or 100% identical to SEQ ID NO: 14, or fragments thereof.

In another aspect, the disclosure provides a PDGFRbeta-Fc fusion protein comprising an amino acid sequence that is at least 90%, 95%, 96%, 97%, 98%, 99% or 100% identical to any one of SEQ ID NOs: 15-25, or biologically active fragments thereof. In some embodiments, PDGFRbeta-Fc fusion protein comprises an amino acid sequence that is at least 90%, 95%, 96%, 97%, 98%, 99% or 100% to SEQ ID NO: 19, or biologically active fragments thereof.

In certain embodiments of any of the foregoing or following, the PDGFRbeta-Fc fusion protein, or biologically active fragment thereof, has an IC₅₀ for phosphorylation of Protein Kinase B (AKT) that is at least 2, 3, 5, 7, 10, 12, 15, 20, 30, 40, 50, 60, 70, 80, 90, 100, 110, 120, 125, 130, 140, 150, 160, 170, 180, 190, or 200 fold lower than that of a linker-less PDGFRbeta-Fc fusion protein in a PDGF-BB-mediated phosphorylation assay. In some embodiments the PDGFRbeta-Fc fusion protein, or biologically active fragment thereof, has an IC₅₀ for phosphorylation of Protein Kinase B (AKT) that is 2-500, 2-200, 2 to 100, 2 to 50 fold, 2 to 25 fold, 5 to 15 fold, or 2 to 10 fold lower than that of a linker-less PDGFRbeta-Fc fusion protein in a PDGF-BB-mediated phosphorylation assay. In other embodiments, the PDGFRbeta-Fc fusion protein, or biologically active fragment thereof, has an IC₅₀ for PDGFRbeta auto-phosphorylation that is at least 2, 3, 5, 6, 7, 8, 9, 10, 11, 12, 13, 14 or 15-fold lower than that of a linker-less PDGFRbeta-Fc fusion protein in a PDGF-BB-mediated phosphorylation assay. In yet other embodiments, the PDGFRbeta-Fc fusion protein has an IC₅₀ for PDGFRbeta auto-phosphorylation that is 2 to 100, 2 to 50 fold, 2 to 25 fold, 5 to 15 fold, or 2 to 5 fold lower than that of a linker-less PDGFRbeta-Fc fusion protein in a PDGF-BB-mediated phosphorylation assay.

In certain embodiments the PDGFRbeta-Fc fusion proteins, or biologically active fragments thereof, of the present disclosure have an intravitreal half-life that is at least 10% to 60%, or 15% to 45%, or 20 to 50%, or 30% to 40% longer than that of a linker-less PDGFRbeta-Fc fusion protein. In certain embodiments of any of the foregoing or following, the PDGFRbeta-Fc fusion protein, or biologically active fragment thereof, has an intravitreal half-life that is at least 30% longer than that of a linker-less PDGFRbeta-Fc fusion protein. In some embodiments the PDGFRbeta-Fc fusion protein, or biologically active fragment thereof, has an intravitreal half-life that is at least 20%, or 25%, or 30%, or 40%, or 50% or 60% or 70% or 80% or 90% or 100% longer than that of a linker-less PDGFRbeta-Fc fusion protein.

Further provided herein is a composition comprising a PDGFRbeta-Fc fusion protein, or biologically active fragment thereof, described in any of the foregoing or following embodiments, and at least one pharmaceutically acceptable carrier or excipient. The composition may be an aqueous formulation for parenteral administration. The composition may contain 1-200 mg/mL, 1-100 mg/ml, 1-50 mg/ml or 1-25 mg/ml of the PDGFRbeta-Fc fusion protein, or biologically active fragment thereof. In particular embodiments, the composition is formulated as eye drops, eye wash, ophthalmic solutions, ophthalmic suspensions, ophthalmic emulsions, ophthalmic ointment, intravitreal injections, subtenon injections, an ophthalmic bioerodible implant, or a non-bioerodible ophthalmic implant.

Also provided herein are isolated nucleic acids encoding any of the PDGFRbeta-Fc fusions proteins, or biologically active fragments thereof, described herein and/or isolated nucleic acids that hybridize under stringent conditions to a nucleic acid sequence selected from any one of SEQ ID NOS: 26-36. In some embodiments, the nucleic acid comprises a sequence that is at least 90%, 95%, 96%, 97%, 98%, 99% or 100% identical to any one of SEQ ID NOs: 26-36. In some embodiments, the nucleic acid is a recombinant nucleic acid, comprising a promoter sequence operably linked to a nucleotide sequence encoding any of the PDGFRbeta-Fc fusions proteins, or biologically active fragments thereof, described herein.

The disclosure also provides vectors and plasmids comprising a nucleic acid sequence encoding any of the PDGFRbeta-Fc fusions proteins, or biologically active fragments thereof, described herein. The nucleic acids, vectors and/or plasmid may be provided in a kit including instructions for transfection and expression in a host cell or may be used in gene therapy.

Also provided herein, is a host cell comprising a nucleic acid encoding a PDGFRbeta-Fc fusion protein, or biologically active fragment thereof, as described herein. In particular embodiments the host cell is a prokaryotic cell. In other embodiments the host cell is a eukaryotic cell.

Another aspect of the disclosure provides methods for treating an ocular neovascular disorder, comprising administering to a subject any of the PDGFRbeta-Fc fusion proteins, or biologically active fragments thereof, described herein. In some embodiments the administration decreases neovascularization. In a further embodiment, the administration of any of the PDGFRbeta-Fc fusion proteins, or biologically active fragments thereof, disclosed herein to a subject having any of the disorders disclosed herein (e.g., AMD) does not substantially increase vascular leakage in contrast to other known anti-PDGF agents. As used herein, “does not substantially increase vascular leakage” means that the vascular leakage score determined by angiography increases by no more than 20%, 15%, 10%, 8%, 5%, or 2% as compared to vascular leakage observed in an untreated control subject. In some embodiments, the administration decreases vascular leakage as compared to the vascular leakage observed in an untreated control subject. In some embodiments, the administration decreases vascular leakage by at least 10%, 20%, 30%, 40%, 50%, 60%, 70%, 80%, 90% or 100% as compared to the vascular leakage observed in an untreated control subject.

In a further aspect the disclosure provides methods for inhibiting ocular neovascularization, comprising administering to a subject any of the PDGFRbeta-Fc fusion proteins, or biologically active fragments thereof, described herein, wherein said administration does not substantially increase vascular leakage as compared to an untreated control subject. In a further embodiment, the administration decreases vascular leakage as compared to an untreated control subject. In some embodiments, the administration decreases neovascularization as compared to an untreated control subject.

In certain embodiments, the ocular neovascular disorder is age-related macular degeneration, choroidal neovascularization, choroidal neovascular membrane, cystoid macula edema, epi-retinal membrane and macular hole, myopia-associated choroidal neovascularisation, vascular streaks, retinal detachment, diabetic retinopathy, diabetic macular edema, atrophic changes of the retinal pigment epithelium, hypertrophic changes of the retinal pigment epithelium, retinal vein occlusion, choroidal retinal vein occlusion, macular edema, macular edema due to retinal vein occlusion, proliferative vitreoretinopathy, familiar exudative vitreoretionpathy, retinitis pigmentosa, Stargardt's disease, retinopathy of prematurity, keratitis, corneal transplantation or keratoplasty, corneal angiogenesis due to hypoxia, pterygium conjunctivae, subretinal edema or intraretinal edema. In some embodiments, the ocular neovascular disorder is wet age-related macular degeneration.

The PDGFRbeta-Fc fusion proteins, or biologically active fragments thereof, may be administered orally, topically, intravitreally, intraocularly, intravenously, subcutaneously, intramuscularly, intraperitoneally, intranasally or peribulbarly. The PDGFRbeta-Fc fusion proteins, or biologically active fragments thereof, may also be administered to the eye topically, by intravitreal injection, or by intraocular insertion of a biodegradable or non-biodegradable drug delivery system.

Another aspect of the disclosure provides a method for treating an ocular neovascular disorder, comprising administering to a subject any of the PDGFRbeta-Fc fusion proteins, or biologically active fragments thereof, described herein; and a receptor tyrosine kinase signaling antagonist. In certain embodiments, the receptor tyrosine kinase antagonist is a VEGF antagonist, IGF antagonist, FGF antagonist or a HGF antagonist. In some embodiments the combined PDGFRbeta-Fc, or biologically active fragment thereof, and receptor tyrosine kinase treatment decreases neovascularization as compared to an untreated control subject or as compared to a subject receiving only a PDGFRbeta-Fc or biologically active fragment thereof treatment or as compared to only a receptor tyrosine kinase treatment. In further embodiments the combined PDGFRbeta-Fc, or biologically active fragment thereof, and receptor tyrosine kinase treatment does not substantially increase vascular leakage or even decreases vascular leakage as compared to an untreated control subject or as compared to a subject receiving only a PDGFRbeta-Fc, or biologically active fragment thereof, treatment or only a receptor tyrosine kinase treatment.

In some embodiments the receptor tyrosine kinase antagonist is a VEGF antagonist. In further embodiments, the VEGF antagonist is a small molecule, an antibody, a VEGF trap, an aptamer, a RNAi construct or an antisense construct. In still further embodiments, the VEGF antagonist is regorafenib, a hydrate, solvate or pharmaceutical acceptable salt thereof or a polymorph thereof. The VEGF antagonist may be provided in a sustained release or eye-drop formulation. In some embodiments, the VEGF antagonist comprises eye-drops or a sustained release depot formulation of regorafenib.

In yet another embodiment, the PDGFRbeta-Fc fusion protein (or biologically active fragment thereof) and/or the receptor tyrosine kinase signaling antagonist are administered orally, topically, intravitreally, intraocularly, intravenously, subcutaneously, intramuscularly, intraperitoneally, intranasally or peribulbarly. In a further embodiment the PDGFRbeta-Fc fusion protein (or biologically active fragment thereof) and/or the receptor tyrosine kinase signaling antagonist are administered to the eye topically, by intravitreal injection, or by intraocular insertion of a biodegradable or non-biodegradable drug delivery system.

In another embodiment, the PDGFRbeta-Fc protein (or biologically active fragment thereof) and the receptor tyrosine kinase signaling antagonist are administered simultaneously followed by additional administration of PDGFRbeta-Fc for three months. In certain embodiments the PDGFRbeta-Fc is given biweekly, monthly or bimonthly for 1, 2, 3, 4, 5, 6, 7, 8 9, 10, 11 or 12 months or indefinitely after the initial treatment.

In some embodiments, the relative increase of in vitro potency measured as IC50 for phosphorylation of Protein Kinase B (AKT) in a PDGF-BB-mediated phosphorylation assay for any of the PDGFRbeta-Fc fusion proteins having a linker described herein as compared to a linker-less PDGFRbeta-Fc fusion protein is at least 2, 3, 4, 5, 6, 7, 8, 9, 10, 12, 14, 16, 18 or 20 times greater than the increase in binding affinity of the PDGFRbeta-Fc fusion protein for the PDGF ligand -BB compared to the linker-less PDGFRbeta-Fc fusion protein.

### BRIEF DESCRIPTION OF THE FIGURES

FIG. 1 outlines the molecules of the disclosure: A) shows a schematic representation of the native PDGFRbeta. B) gives a schematic view of PDGFRbeta-Fc fusion protein with a given linker. 1-5=IgG domains; T=transmembrane domain; K=kinase domain; L=linker; Fc=Fc part of an antibody; C=cell membrane.

FIG. 2 summarizes the IC₅₀ values (y-axis in both (A) and (B) is [M]) of PDGFRbeta-Fc constructs (x-axis) in cell-based assays on the Meso Scale ELISA platform. A) shows data obtained with the pPDGFRbeta Meso Scale assay, and B) shows data obtained with the pAKT Meso Scale assay. PDGFRbetaln the pAKT assay reduction of the IC₅₀ values by addition of a linker was even more pronounced, highlighted by a reduction 129 fold lower IC₅₀ value for the 5×GGGGS linker variant. The IC₅₀ values for the variants carrying a linker of n>3×GGGGS were not statistically different from each other.

FIG. 3 shows different treatment protocols for the rat laser-induced choroidal neovascularization model. A) Early treatment protocol—the therapeutic agent is applied as a single intravitreal injection on the day after laser-induced disruption of Bruch's membrane which induces a neovascular lesion through choroidea and retina. Following a fluorescence angiography vascular leakage is graded at day 21 post laser on a scale from 0 to 3, with grade 0 for no fluorescence and grade 3 for strong fluorescence at the lesion site. Neoangiogenesis is determined at day 23 post laser treatment by immunofluorescence staining of vessels using a CD31 antibody and microscopic measurement of the neovascular area. B) Delayed short treatment protocol—the therapeutic agent is injected intravitreally at day 7 post laser treatment. Vascular leakage and angiogenesis are determined at day 14 and 16, respectively. C) Delayed long treatment protocol—the therapeutic agent is injected intravitreally at day 7 post laser treatment. Vascular leakage and angiogenesis are determined at day 21 and 23, respectively.

FIG. 4 shows a comparison of the in vivo activity of a linker-less PDGFRbeta-Fc fusion protein with a PDGF-B aptamer in the rat laser CNV model, early treatment protocol. A) The y-axis is the “relative neovascular area” shown as a percentage of the relative neovascular area of untreated control animals. The PDGFRbeta-Fc fusion protein shows a stronger reduction in neovascularization than a PDGF-B aptamer. B) The y-axis is the “vascular leakage score” determined by angiography and is shown in [%] of the 21 d baseline leakage. In contrast to the PDGF-B aptamer, which increases vascular leakage, the vascular leakage score is not affected by a PDGFRbeta-Fc fusion protein in this treatment setting.

FIG. 5 shows the in vivo activity of PDGFRbeta-Fc constructs carrying no linker or a 3×GGGGS linker in the delayed short treatment protocol. A) Both PDGFRbeta-Fc constructs reduce relative neovascularization (y-axis [%]) compared to untreated control and anti-VEGF positive control animals. B) The vascular leakage score (y-axis) is shown in [%] of the 7 d baseline leakage. In this treatment protocol, the 3×GGGGS linker variant of the PDGFRbeta-Fc fusion protein shows a reduction in vascular leakage that is more pronounced than in the anti-VEGF control group. The PDGFRbeta-Fc variant without linker shows even a slight increase in vascular leakage. This demonstrates that the higher in vitro activity of PDGFRbeta-Fc fusion protein variants translates into in vivo activity that improves the therapeutic effect against CNV and the associated vision impairing vascular leakage.

FIG. 6 shows the combination effects of anti-VEGF and PDGFRbeta-Fc fusion protein with a 4×GGGGS linker in the delayed long treatment protocol. A) Anti-VEGF and the 4×GGGGS linker PDGFRbeta-Fc fusion protein show a reduction in the relative neovascular area (y-axis [%]) compared to untreated control animals with laser induced lesions either alone or in combination. An additive effect cannot be observed likely due to a ceiling effect in this experimental setting. B) Both, anti-VEGF and 4×GGGGS linker PDGFRbeta-Fc fusion protein reduce the vascular leakage score (y-axis) compared to untreated control animals. Here, the combination of both agents shows an additive effect on reduction of vascular leakage underpinning the mechanism of vessel re-sensitization towards anti-VEGF treatment regimens by blockade of PDGF signaling.

FIG. 7 shows the PK profile in the rabbit vitreous humor after single intravitreal application of a PDGFRbeta-Fc fusion protein without linker (dashed line, triangles) and a 4×GGGGS linker variant (solid line, circles). The concentration (y-axis in [μg/L]) of the 4×GGGGS linker variant declines less rapidly than that of the no linker variant (x-axis in [h]), resulting in an increased vitreal half-life of 7.21 d for the 4×GGGGS linker variant versus 4.75 d for the PDGFRbeta-Fc construct without linker.

FIG. 8A-80 depict the Sequence ID NOs 1-36.

### DETAILED DESCRIPTION OF THE DISCLOSURE

### Definitions

Unless defined otherwise, all technical and scientific terms used herein have the meaning commonly understood by one of ordinary skill in the art to which this disclosure belongs. The following references, however, can provide one of skill in the art to which this disclosure pertains with a general definition of many of the terms used in this disclosure, and can be referenced and used so long as such definitions are consistent with the meaning commonly understood in the art. Such references include, but are not limited to, Singleton et al, Dictionary of Microbiology and Molecular Biology (2d ed. 1994); The Cambridge Dictionary of Science and Technology (Walker ed., 1988); Hale & Marham, The Harper Collins Dictionary of Biology (1991); and Lackie et al., The Dictionary of Cell & Molecular Biology (3d ed. 1999); and Cellular and Molecular Immunology, Eds. Abbas, Lichtman and Pober, 2nd Edition, W.B. Saunders Company. Any additional technical resources available to the person of ordinary skill in the art providing definitions of terms used herein having the meaning commonly understood in the art can be consulted. For the purposes of the present disclosure, the following terms are further defined. Additional terms are defined elsewhere in the description. As used herein and in the appended claims, the singular forms “a,” “and,” and “the” include plural reference unless the context clearly dictates otherwise. Thus, for example, reference to “a gene” is a reference to one or more genes and includes equivalents thereof known to those skilled in the art, and so forth.

The term “PDGF” relates to the Platelet-Derived Growth Factor (PDGF) family of ligands of the two receptor tyrosine kinases PDGFRbeta and PDGFRalpha. The PDGF family consists of four members (A, B, C, D) that share a cluster of 8 cysteine residues. PDGFs form biologically active homodimers (PDGF-AA, PDGF-BB, PDGF-CC, PDGF-DD) and one heterodimer (PDGF-AB) via disulfide bonds. PDGFRalpha and PDGFRbeta, homo- or hetero-dimerize upon ligand binding. The specificity of the different PDGF ligands for both receptors varies, with PDGF-BB and -DD binding PDGFRbeta, and PDGF-BB, PDGF-CC and -AA binding PDGFRalpha. PDGF-BB, PDGF-AB and to a weaker extent PDGF-CC and -DD were also reported to bind and activate the heterodimeric PDGFRalpha/beta (Heldin and Westermark, Physiol Rev 1999; 79(4): 1283-1316; Reigstad et al., FEBS J 2005; 272: 5723-5741).

The term “PDGFRbeta” refers to the platelet-derived growth factor receptor beta, a 180-kDa transmembrane glycoprotein which binds PDGF BB with high affinity (Duan et al., J Biol Chem. 266: 413-418 1991). This type I transmembrane glycoprotein comprises five NH2-terminal Ig domains, the extracellular domains D1-D5. It is anchored in the membrane by a single transmembrane domain and contains an intracellular receptor tyrosine kinase domain making it a member of the receptor tyrosine kinase family. The extracellular domain of PDGFRbeta or fragments thereof are capable of binding one or more of the PDGF ligands -AA, -AB, -BB, -CC and -DD thereby blocking biological activities mediated by these ligands, for example its effects on vessel maturation. Unless explicitly stated otherwise, this term encompasses any of the PDGFRbeta variants or biologically active fragments disclosed herein. In certain embodiments, PDGFRbeta comprises an amino acid sequence that is at least 90%, 95%, 96%, 97%, 98%, 99% or 100% identical to SEQ ID NO: 1, or biologically active fragments thereof.

The terms “PDGFRbeta-Fc”, “PDGFRbeta-Fc fusion protein” and “PDGFRbeta-Fc protein” are defined as fusion proteins comprising a PDGFRbeta portion and a Fc portion. Unless explicitly stated otherwise, the term “PDGFRbeta-Fc”, “PDGFRbeta-Fc fusion protein” or “PDGFRbeta-Fc protein” encompasses any of the PDGFRbeta-Fc protein variants or biologically active fragments disclosed herein. In certain embodiments, the extracellular domain of a PDGFRbeta-Fc fusion protein comprises an amino acid sequence that is at least 90%, 95%, 96%, 97%, 98%, 99% or 100% identical to any of SEQ ID NOs: 15-25, or biologically active fragments thereof.

The “extracellular ligand binding domain, or “extracellular domain” is defined as the portion of a receptor that, in its native conformation in the cell membrane, is oriented extracellularly where it can contact with its cognate ligand. The extracellular ligand binding domain does not include the hydrophobic amino acids associated with the receptor's transmembrane region or any amino acids associated with the receptor's intracellular part. The preceding 15-30, predominantly hydrophobic or apolar amino acids (i.e., leucine, valine, isoleucine, and phenylalanine) comprise the transmembrane region. The extracellular domain comprises the amino acids that precede the hydrophobic transmembrane stretch of amino acids. von Heijne has published detailed rules that are commonly referred to by skilled artisans when determining which amino acids of a given receptor belong to the extracellular, transmembrane, or intracellular parts (cf. von Heijne (1995) BioEssays 17:25). In certain embodiments, the extracellular domain of PDGFRbeta comprises an amino acid sequence that is at least 90%, 95%, 96%, 97%, 98%, 99% or 100% identical to SEQ ID NO: 2 or 3, or biologically active fragments thereof.

The terms “Fc”, “Fc-part”, Fc-region, Fc-portion or “Fc-domain” denote the carboxy-terminal region of an immunoglobulin heavy chain of mammalian origin. As is known, the immunoglobulin heavy chain constant region comprises three or four domains and a hinge region. The domains are named sequentially as follows: CH1-hinge-CH2-CH3(-CH4). The DNA sequences of the heavy chain domains have cross-homology among the immunoglobulin classes, e.g., the CH2 domain of IgG is homologous to the CH2 domain of IgA and IgD, and to the CH3 domain of IgM and IgE. As used herein, the terms, “Fc”, “Fc-part” or “Fc domain” are understood to mean the carboxyl-terminal portion that contains at least a part of the hinge region, the CH2 domain and the CH3 domain. In one embodiment, the class of immunoglobulin from which the Fc-part is derived is IgG (Igγ) (γ subclasses 1, 2, 3, or 4). Other classes of immunoglobulin, IgA (Igα), IgD (Igδ), IgE (Igε) and IgM (Igμ), may be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. In one embodiment, a human IgG1 heavy chain Fc-region extends from Asp 221, Cys226, or from Pro230, to the carboxyl-terminus of the heavy chain. In one embodiment the Fc-region has the amino acid sequence of SEQ ID NO: 14. However, the C-terminal lysine (Lys447) of the Fc-region may or may not be present. Unless otherwise specified herein, numbering of amino acid residues in the Fc-region or constant region is according to the EU numbering system, also called the EU index, as described in Kabat, E. A., et al, Sequences of Proteins of Immunological Interest, 5th ed., Public Health Service, National Institutes of Health, Bethesda, Md. (1991), NIH Publication. Furthermore, it is contemplated that substitution or deletion of amino acids within the immunoglobulin heavy chain constant regions may be useful in the practice of the disclosure. A “Fc part of an antibody” is a term well known to the skilled artisan and defined on the basis of papain cleavage of antibodies. A “fusion protein” as used herein refers to an expression product resulting from the fusion of at least two genes. An “Fc-fusion protein” or “Fc-fusion” is a chimeric polypeptide comprising the Fc-region, or constant region, of an antibody fused, or conjugated, to an unrelated protein or protein fragment either at the C- or the N-terminus or even both termini of the Fc part. In certain embodiments, the Fc comprises an amino acid sequence that is at least 90%, 95%, 96%, 97%, 98%, 99% or 100% identical to SEQ ID NO: 14.

A “biologically active fragment thereof” is a fragment of protein or peptide that retains at least 10%, at least 20%, at least 30%, at least 40%, at least 50%, at least 60%, at least 70%, at least 80%, at least 90% or at least 99% of the biological activity of the reference protein or peptide. In some embodiments, a biologically active fragment of a PDGFRbeta or PDGFRbeta-Fc protein binds one or more of PDGF ligands -BB, -DD, and -AB with a K_{D} of less than 300 nM, less than 250 nM, less than 200 nM, less than 150 nM, less than 100 nM, less than 50 nM, less than 10 nM, less than 1 nM, less than 500 pM, less than 200 pM, less than 125 pM, less than 100 pM, less than 50 pM, less than 30 pM or less than 10 pM. In some embodiments, a biologically active fragment of a PDGFRbeta or PDGFRbeta-Fc protein binds one or more of PDGF ligands -BB, -DD, and -AB with a K_{D} of between 1-100 pM, between 100-500 pM, between 500-1000 pM, between 1 nM and 100 nM, or between 100-500 nM. In some embodiments, a biologically active fragment of a PDGFRbeta or PDGFRbeta-Fc protein binds one or more of the PDGF ligands -BB and -DD with a K_{D} of less than 200 pM, or less than 125 pM, or less than 100 pM, or less than 50 pM, or less than 30 pM. In some embodiments, a biologically active fragment of a PDGFRbeta or PDGFRbeta-Fc protein binds one or more of the PDGF ligands -BB and -DD with a K_{D} of 1 to 500 pM, 1 to 200 pM, 1 to 100 pM, 1 to 50 pM, 50 to 150 pM or 100 to 200 pM.

A “linker” refers to an amino acid sequence which is used to connect or fuse two or more different proteins or two or more different protein domains. “Linkers” of this disclosure are used to link a PDGFRbeta part to a Fc-part to form an PDGFRbeta fusion protein according to the disclosure.

“Binding affinity” refers to the strength of the sum total of non-covalent interactions between a single binding site of a molecule and its binding partner. Unless indicated otherwise, as used herein, “binding affinity” refers to intrinsic binding affinity which reflects a 1:1 interaction between members of a binding pair (e.g. a receptor or a receptor fusion protein according to the disclosure and a ligand). The dissociation constant “K_{D}” is commonly used to describe the affinity between a molecule (such as receptor or a receptor fusion protein according to the disclosure) and its binding partner (such as a ligand) i.e. how tightly a ligand binds to a particular protein. Ligand-protein affinities are influenced by non-covalent intermolecular interactions between the two molecules. Affinity can be measured by common methods known in the art, including those described herein. In one embodiment, the “K_{D}” or “K_{D} value” according to this disclosure is measured by using surface plasmon resonance assays using a Biacore T200 instrument (GE Healthcare Biacore, Inc.) with Series S Sensor Chips CM5. In brief, binding assays were performed at 25° C. with assay buffer HBS-EP+(including 1 mg/ml BSA, 500 nM NaCl, 0.05% NaN₃ in total). Fc fusions were captured with an anti-hIgG capture-Ab covalently coupled to the chip surface via amine coupling chemistry. Subsequently, PDGF antigens were used as analyte in various concentrations and after each association and dissociation phase the chip surface was regenerated (glycine HCl pH 2.0), followed by another capture and analyte injection cycle. Obtained sensorgrams were double-referenced, i.e. in-line reference cell correction followed by buffer sample subtraction. K_{D} values for PDGF-BB and -DD were calculated based on the ratio of dissociation (k_{d}) and association (kₐ) rate constants which were obtained by globally fitting sensorgrams with a first order 1:1 Langmuir binding model. Data for PDGF-AB were evaluated by a steady-state affinity plot. Other suitable instruments are e.g. Biacore 2000, a Biacore 3000, Biacore 4000 or Biacore T100 (GE Healthcare Biacore, Inc.), ProteOn XPR36 instrument (Bio-Rad Laboratories, Inc.), IBIS MX96 (IBIS Technologies B.V.), or similar.

“Percent (%) sequence identity” with respect to a reference polynucleotide or polypeptide sequence, respectively, is defined as the percentage of nucleic acid or amino acid residues, respectively, in a candidate sequence that are identical with the nucleic acid or amino acid residues, respectively, in the reference polynucleotide or polypeptide sequence, respectively, after aligning the sequences and introducing gaps, if necessary, to achieve the maximum percent sequence identity. Conservative substitutions are not considered as part of the sequence identity. In some embodiments, the alignments are un-gapped alignments. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. In some embodiments the Fc fusion protein is “isolated”. An isolated biological component (such as a nucleic acid molecule or protein such as an antibody or Fc fusion) is one that has been substantially separated or purified away from other biological components in the cell of the organism in which the component naturally occurs, e.g., other chromosomal and extra-chromosomal DNA and RNA, proteins and organelles. Nucleic acids and proteins that have been “isolated” include nucleic acids and proteins purified by standard purification methods as described for example in J. Sambrook et al., 1989 (Molecular Cloning: A laboratory manual, Cold Spring Harbor Laboratory Press, Cold Spring Harbor, USA) and R. K. Scopes et al. 1994 (Protein Purification,—Principles and Practice, Springer Science and Business Media LLC). The term also embraces nucleic acids and proteins prepared by recombinant expression in a host cell as well as chemically synthesized nucleic acids.

The term “subject” as used herein means an animal, including humans and non-human animals. In some embodiments, the subject is a human.

The term “patient” as used herein means a subject having any of the disorders disclosed herein.

The term “antagonist” as used herein means a molecule that blocks, reduces or inhibits the expression of a protein of interest. An antagonist may also mean a molecule that blocks, reduces, or inhibits one or more natural activities of a protein of interest. Antagonism of a receptor can also include reduction or making unavailable the ligands that would otherwise stimulate said receptor. Antagonists include, but are not limited to small molecules, peptides, antibodies, receptor fusion proteins, aptamers, RNAi constructs and antisense constructs.

A “receptor tyrosine kinase signaling antagonist” or “RTK signaling antagonist” or “RTK antagonist” herein means a molecule that blocks, reduces or inhibits the expression or activity of any component in a receptor tyrosine kinase signaling cascade, including the transmembrane receptor with tyrosine kinase activity and downstream effector molecules or proteins. RTK antagonists include, but are not limited to small molecules, peptides, antibodies, receptor fusion proteins, aptamers, RNAi constructs and antisense constructs.

### PDGFRbeta-Fc Fusion Proteins of the Disclosure

The present disclosure relates to PDGFRbeta-Fc fusion proteins and methods for treating pathological neovascularization and fibrosis, e.g. cancer, ocular neovascular disorders, pulmonary fibrosis or nephropathies by providing PDGFRbeta-Fc fusion proteins. The PDGFR-Fc fusion proteins of the present disclosure comprise an extracellular domain of PDGFRbeta and an Fc domain.

In some embodiments, the disclosure provides a PDGFRbeta-Fc fusion protein comprising an extracellular domain of PDGFRbeta and a Fc domain, wherein the PDGFRbeta-Fc fusion protein binds one or more of PDGF ligands -BB, -DD, and -AB with a K_{D} of less than 300 nM, less than 250 nM, less than 200 nM, less than 150 nM, less than 100 nM, less than 50 nM, less than 10 nM, less than 1 nM, less than 500 pM, less than 200 pM, less then 125 pM, less than 100 pM, less than 50 pM, less than 30 pM or less than 10 pM. In some embodiments, the PDGFRbeta-Fc fusion protein binds one or more of PDGF ligands -BB, -DD, and -AB with a K_{D} of between 1-100 pM, between 100-500 pM, between 500-1000 pM, between 1 nM and 100 nM, or between 100-500 nM. In preferred embodiments, the PDGFRbeta-Fc fusion binds one or more of the PDGF ligands -BB, -DD, and -AB with a K_{D} of less than 125 pM. In other preferred embodiments, the PDGFRbeta-Fc fusion protein binds the PDGF ligand -BB with a K_{D} of less than 30 pM. In further preferred embodiments, the PDGFRbeta-Fc fusion binds the PDGF ligands -BB and -DD with a K_{D} of less than 30 pM, respectively.

In some embodiments, the PDGFRbeta-Fc fusion protein does not substantially bind to platelet-derived growth factor ligands AA and CC. For example, the PDGFRbeta-Fc fusion protein may bind to PDGF ligands AA or CC with no meaningful binding affinity, such as it may bind to the ligands AA and CC with a K_{D} of greater than 300 nM, greater than 400 nM, greater than 500 nM, or greater than 1 μM.

In some embodiments, the PDGFRbeta extracellular domain of the fusion protein binds one or more of the PDGF ligands -BB and -DD with a K_{D} of less than 200 pM, or less than 125 pM, or less than 100 pM, or less than 50 pM, or less than 30 pM. In some embodiments, the PDGFRbeta extracellular domain of the fusion protein binds one or more of the PDGF ligands -BB and -DD with a K_{D} of 1 to 500 pM, 1 to 200 pM, 1 to 100 pM, 1 to 50 pM, 50 to 150 pM or 100 to 200 pM.

Toward these ends, it is an embodiment of the invention to provide PDGFRbeta-Fc fusion protein that comprise an N-terminal fragment of PDGFRbeta (SEQ ID NO: 1), wherein the PDGFRbeta fragment comprises amino acids 1-500, 1-400, 1-300, 1-285 or 1-282 of SEQ ID NO: 1, and further wherein the fragment is able to bind one or more PDGF ligands -AA, -AB, -BB, -CC or -DD. In certain embodiments, the N-terminal fragment is a biologically active fragment.

It is another embodiment of the invention to provide fusion proteins that comprise one or more extracellular domains of human PDGFRbeta (SEQ ID NO: 1), selected from D1, D2, D3, D4 and D5. In some embodiments, it comprises the extracellular domains D1-D5 (EDC 1-5) of human PDGFRbeta as set forth in SEQ ID NO: 2. In other embodiments, the PDGFRbeta portion of the fusion protein comprises the extracellular domains D1-D3 (EDC 1-3) of PDGFRbeta (SEQ ID NO: 3), or a biologically active fragment thereof. In other embodiments, the PDGFRbeta portion of the fusion protein comprises an amino acid sequence that is at least 80%, 85%, 90%, 95%, 96%, 97%, 98%, 99% or 100% identical to SEQ ID NO: 2, or a biologically active fragment thereof. In yet other embodiments, the PDGFRbeta portion of the fusion protein comprises an amino acid sequence that is at least 80%, 85%, 90%, 95%, 96%, 97%, 98%, 99% or 100% identical to SEQ ID NO: 3, or a biologically active fragment thereof.

In some embodiments, the PDGFRbeta-Fc fusion proteins, or biologically active fragments thereof, according to the disclosure contain an Fc part derived from mammalian origin, e. g. but not limited to mouse, rat, monkey, pig or human. In some embodiments, the Fc-part is a human Fc and may be from human IgG1, IgG2, IgG3 or IgG4 subclass. In some embodiments, the Fc is derived from human IgG1 subclass, e.g. as outlined in SEQ ID NO: 14. Thus, the Fc-portion may comprise an amino acid sequence that is at least 80%, 85%, 90%, 95%, 96%, 97%, 98%, 99% or 100% identical to SEQ ID NO: 14, or fragments thereof. In one embodiment, the class of immunoglobulin from which the Fc-part is derived is IgG (Igγ) (γ subclasses 1, 2, 3, or 4). Other classes of immunoglobulin, IgA (Igα), IgD (Igδ), IgE (Igε) and IgM (Igμ), may be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. In one embodiment, a human IgG1 heavy chain Fc-region extends from Asp 221, Cys226, or from Pro230, to the carboxyl-terminus of the heavy chain. In one embodiment the Fc-region has the amino acid sequence of SEQ ID NO: 14. However, the C-terminal lysine (Lys447) of the Fc-region may or may not be present. Unless otherwise specified herein, numbering of amino acid residues in the Fc-region or constant region is according to the EU numbering system, also called the EU index, as described in Kabat, E. A., et al, Sequences of Proteins of Immunological Interest, 5th ed., Public Health Service, National Institutes of Health, Bethesda, Md. (1991), NIH Publication. Furthermore, it is contemplated that substitution or deletion of amino acids within the immunoglobulin heavy chain constant regions may be useful in the practice of the disclosure.

In some embodiments, the Fc-portion comprises hybrid heavy chain constant regions, i.e., the Fc-portion comprises multiple heavy chain constant region domains selected from: a CH1 domain, a CH2 domain, a CH3 domain, and a CH4 domain; wherein at least one of the constant region domains in the Fc is of a class or subclass of immunoglobulin distinct from the class or subclass of another domain in the Fc. In some embodiments, at least one of the constant region domains in the Fc is an IgG constant region domain, and at least one of the constant region domains in the Fc is of a different immunoglobulin class, i.e., an IgA, IgD, IgE, or IgM constant region domain. In some embodiments, at least one of the constant region domains in the Fc is a IgG1 constant region domain, and at least one of the constant region domains in the Fc is of a different IgG subclass, i.e., IgG2, IgG2, IgG3 or IgG4. Suitable constant regions may be human or from another species (e.g., murine).

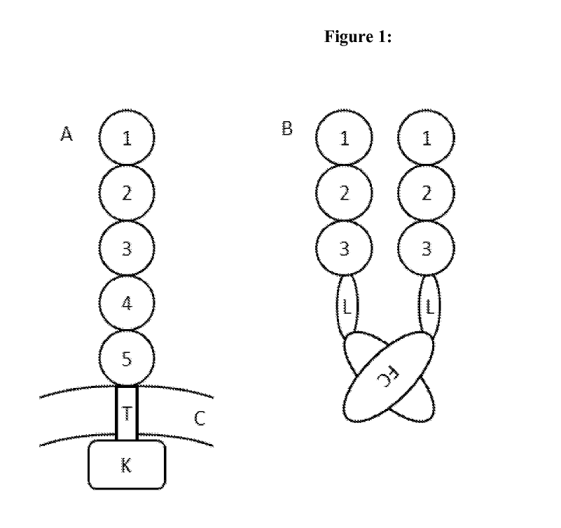

In a further embodiment, the PDGFRbeta-Fc fusion proteins of the disclosure are fusions of the extracellular domains D1-D3 of PDGFRbeta, derived from UNIPROT ID P09619 (SEQ ID NO: 2 and FIG. 1A) to a human IgG1 Fcpart (SEQ ID NO: 14) connected via the C-terminal amino acid of the extracellular domain 3 to the N-terminal amino acid of the Fc-part. The connection can either be done directly (e.g., SEQ ID NO: 15; linker-less PDGFRbeta-Fc fusion protein) or via different amino acid linkers (e.g., SEQ ID NOs: 4-13) to generate the PDGFRbeta-Fc fusion proteins as outlined in SEQ ID NOs: 16-25. The disclosure also contemplates PDGFRbeta-Fc fusion proteins comprising an amino acid sequence that is at least 80%, 85%, 90%, 95%, 96%, 97%, 98%, 99% or 100% identical to any one of SEQ ID NOs: 15-25, or biologically active fragments thereof.

In some embodiments, the PDGFRbeta portion is connected to the Fc portion by means of a linker. In some embodiments, the linkers are glycine and serine rich linkers. Other near neutral amino acids, such as, but not limited to, Thr, Asn, Pro and Ala, may also be used in the linker sequence. In some embodiments, the linker comprises various permutations of amino acid sequences containing Gly and Ser. In further embodiments, the linkers have a length of at least 12, 15, 20, 25, 30, 35, 40, 45 or 50 amino acids. In preferred embodiments, the linker comprises the amino acid sequence GlyGlyGlyGlySer (GGGGS), or repetitions thereof (GGGGS)n, where n≧1. In more preferred embodiments n≧3, or n=3-10. In even more preferred embodiments n=3-7, or n=3, 4, 5, 6, or 7. In further preferred embodiments, n=4.

Examples of linkers according to the disclosure are outlined in SEQ ID NO: 7-13, i.e.

SEQ ID NO 7:
GGGGS (1xGGGGS),
SEQ ID NO 8:
GGGGSGGGGS (2xGGGGS),
SEQ ID NO 9:
GGGGSGGGGSGGGGS (3xGGGGS),
SEQ ID NO 10:
GGGGSGGGGSGGGGSGGGGS (4xGGGGS),
SEQ ID NO 11:
GGGGSGGGGSGGGGSGGGGSGGGGS (5xGGGGS),
SEQ ID NO 12:
GGGGSGGGGSGGGGSGGGGSGGGGSGGGGS (6xGGGGS),
SEQ ID NO 13:
GGGGSGGGGSGGGGSGGGGSGGGGSGGGGSGGGGS (7xGGGGS).

In a further embodiment the PDGFRbeta-Fc fusion proteins of the present disclosure have an IC₅₀ for phosphorylation of Protein Kinase B (AKT) that is 2-500, 2-200, 2 to 100, 2 to 50 fold, 2 to 25 fold, 5 to 15 fold, or 2 to 10 fold lower than that of a linker-less PDGFRbeta-Fc fusion protein in a PDGF-BB-mediated phosphorylation assay. In some embodiments, a fusion protein of the disclosure has an IC₅₀ for phosphorylation of Protein Kinase B (AKT) that is at least 2, 3, 5, 7, 10, 12, 15, or 20, 30, 40, 50, 60, 70, 80, 90, 100, 110, 120, 125, 130, 140, 150, 160, 170, 180, 190, or 200 fold lower than that of a linker-less PDGFRbeta-Fc fusion protein in a PDGF-BB-mediated phosphorylation assay. In a preferred embodiment the PDGFRbeta-Fc fusion proteins of the present disclosure have an IC₅₀ for phosphorylation of Protein Kinase B (AKT) that is at least 10-fold lower than that of a linker-less PDGFRbeta-Fc fusion protein in a PDGF-BB-mediated phosphorylation assay.

In a further embodiment the PDGFRbeta-Fc fusion proteins of the present disclosure also have an IC₅₀ for PDGFRbeta auto-phosphorylation that 2 to 100, 2 to 50 fold, 2 to 25 fold, 5 to 15 fold, 2 to 5, or 3 fold lower than that of a linker-less PDGFRbeta-Fc fusion protein in a PDGF-BB-mediated phosphorylation assay. In some embodiments, a fusion protein of the disclosure has an IC₅₀ for PDGFRbeta auto-phosphorylation that is at least 2, 3, 4, 5, 6, 7, 8, 9, 10, 11, 12, 13, 14 or 15 fold lower than that of a linker-less PDGFRbeta-Fc fusion protein in a PDGF-BB-mediated phosphorylation assay.

In a further embodiment the PDGFRbeta-Fc fusion proteins of the present disclosure have an intravitreal half-life that is at least 10% to 60%, or 15% to 45%, or 20 to 50%, or 30% to 40% longer than that of a linker-less PDGFRbeta-Fc fusion protein. In some embodiments, a fusion protein of the disclosure has an intravitreal half-life that is at least 20%, 30%, 40%, 50%, 60%, 70%, 80%, 90% or 100% longer than that of a linker-less PDGFRbeta-Fc fusion protein. In some embodiments, PDGFRbeta-Fc fusion proteins of the present disclosure are labeled with a detectable moiety. In some embodiments, the detectable moiety is a fluorophore, radioisotope, enzymatic label, chemiluminescent label, or biotin label. In some embodiments, the PDGFRbeta-Fc fusion protein is conjugated to an epitope tag.

In some embodiments, the relative increase of in vitro potency measured as IC50 for phosphorylation of Protein Kinase B (AKT) in a PDGF-BB-mediated phosphorylation assay for any of the PDGFRbeta-Fc fusion proteins having a linker described herein as compared to a linker-less PDGFRbeta-Fc fusion protein is at least 2, 3, 4, 5, 6, 7, 8, 9, 10, 12, 14, 16, 18 or 20 times greater than the increase in binding affinity of the PDGFRbeta-Fc fusion protein for the PDGF ligand -BB compared to the linker-less PDGFRbeta-Fc fusion protein. In a preferred embodiment the relative increase of in vitro potency measured as IC50 for phosphorylation of Protein Kinase B (AKT) in a PDGF-BB-mediated phosphorylation assay for any of the PDGFRbeta-Fc fusion proteins having a linker described herein as compared to a linker-less PDGFRbeta-Fc fusion protein is at least 6 times greater than the increase in binding affinity of the PDGFRbeta-Fc fusion protein for the PDGF ligand -BB compared to the linker-less PDGFRbeta-Fc fusion protein.

### Peptide Variants

PDGFRbeta-Fc fusion proteins of the disclosure are not limited to the specific peptide sequences provided herein. Rather, the disclosure also embodies variants of these polypeptides. With reference to the present disclosure and conventionally available technologies and references, the skilled worker will be able to prepare, test and utilize functional variants of the PDGFRbeta-Fc fusion proteins disclosed herein, while appreciating that such variants having the ability to bind to PDGF ligands fall within the scope of the present disclosure. Accordingly, the disclosure contemplates PDGFRbeta-Fc fusion proteins comprising an amino acid sequence that is at least 80%, 85%, 90%, 95%, 96%, 97%, 98%, 99% or 100% identical to any one of SEQ ID NOs: 15-25, or biologically active fragments thereof.

A variant can include, for example, a PDGFRbeta-Fc fusion protein that has at least one altered PDGFRbeta domain and/or Fc domain and/or linker sequence, vis-à-vis a peptide sequence disclosed herein.

Furthermore, variants may be obtained by using a gene encoding one PDGFRbeta or fusion protein thereof as starting point for optimization by diversifying one or more codons encoding amino acid residues in the PDGFRbeta-Fc fusion protein, and by screening a resulting collection of PDGFRbeta-Fc fusion protein variants for variants with improved properties. Diversification can be done by synthesizing a collection of DNA molecules using DNA mutagenesis techniques, which are well known in the art, for example “saturation mutatgenesis” techniques that rely on trinucleotide mutagenesis (TRIM) technology (Virnekas B. et al., Nucl. Acids Res. 1994, 22: 5600). PDGFRbeta-Fc fusion proteins include molecules with modifications/variations including but not limited to modifications leading to altered pharmacokinetics (e.g. modification of the Fc part or attachment or removal of further molecules such as PEG or sialic acids), altered binding affinity, altered stability, ligand specifity, or altered potency.

### Conservative Amino Acid Variants

In some embodiments, polypeptide variants may be made that conserve the overall molecular structure of a PDGFRbeta-Fc fusion protein amino acid sequence described herein. Given the properties of the individual amino acids, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved. In some embodiments, the polypeptide variants comprise at least 1, 2, 3, 4, 5, 6, 7, 8, 9, 10, 11, 12, 13, 14, 15, 20, 25, 30, 35, 40, 45, or 50 conservative substitutions.

For example, (a) nonpolar (hydrophobic) amino acids include alanine, leucine, isoleucine, valine, proline, phenylalanine, tryptophane, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt α-helices. Similarly, certain amino acids, such as alanine, cysteine, leucine, methionine, glutamic acid, glutamine, histidine and lysine are more commonly found in α-helices, while valine, isoleucine, phenylalanine, tyrosine, tryptophan and threonine are more commonly found in β-pleated sheets. Glycine, serine, aspartic acid, asparagine, and proline are commonly found in turns. In some embodiments, substitutions may be made among the following groups: (i) S and T; (ii) P and G; and (iii) A, V, L and I. Given the known genetic code and recombinant and synthetic DNA techniques, the skilled scientist readily can construct DNAs encoding the conservative amino acid variants. Alternatively, selected, random or complete subsets of amino acids can be selected at one or more positions in the protein that include non-conservative amino acid substitutions, and a subset of amino acid changes can be incorporated into the final protein that still allow adequate or even enhanced properties.

As used herein, “sequence identity” between two polypeptide sequences, indicates the percentage of amino acids that are identical between the sequences. “Sequence homology” indicates the percentage of amino acids that either is identical or that represent conservative amino acid substitutions.

### Nucleic Acids of the Disclosure

The present disclosure also relates to the nucleic acid molecules (also referred to as polynucleotides herein) that encode a PDGFRbeta-Fc fusion protein of the disclosure. In some embodiments, a nucleic acid molecule that encodes a PDGFRbeta-Fc fusion protein of the disclosure comprises a sequence selected from SEQ ID NOs: 26-36, or fragments thereof that encode biologically active fragments of PDGFRbeta-Fc fusion protein. In some embodiments, the nucleic acids or polynucleotides are DNA. In other embodiments, the nucleic acids or polynucleotides are RNA.

Nucleic acid molecules of the disclosure are not limited to the sequences disclosed herein, but also include variants thereof. Accordingly, in some embodiments, a nucleic acid molecule that encodes a PDGFRbeta-Fc fusion protein of the disclosure comprises a sequence that is at least 80%, 85%, 90%, 95%, 96%, 97%, 98%, 99% or 100% identical to any one of SEQ ID NOs: 26-36, or fragments thereof that encode biologically active fragments of PDGFRbeta-Fc fusion protein.

In some embodiments, nucleic acids, such as DNA variants, within the disclosure may be described by reference to their physical properties in hybridization. The skilled worker will recognize that DNA can be used to identify its complement and, since DNA is double stranded, its equivalent or homolog, using nucleic acid hybridization techniques. It also will be recognized that hybridization can occur with less than 100% complementarity. However, given appropriate choice of conditions, hybridization techniques can be used to differentiate among DNA sequences based on their structural relatedness to a particular probe. For guidance regarding such conditions see, Sambrook et al., 1989 supra and Ausubel et al., 1995 (Ausubel, F. M., Brent, R., Kingston, R. E., Moore, D. D., Sedman, J. G., Smith, J. A., & Struhl, K. eds. (1995). Current Protocols in Molecular Biology. New York: John Wiley and Sons).

Structural similarity between two polynucleotide sequences can be expressed as a function of “stringency” of the conditions under which the two sequences will hybridize with one another. As used herein, the term “stringency” refers to the extent that the conditions disfavor hybridization. Stringent conditions strongly disfavor hybridization, and only the most structurally related molecules will hybridize to one another under such conditions. Conversely, non-stringent conditions favor hybridization of molecules displaying a lesser degree of structural relatedness. Hybridization stringency, therefore, directly correlates with the structural relationships of two nucleic acid sequences. The following relationships are useful in correlating hybridization and relatedness (where Tₙ, is the melting temperature of a nucleic acid duplex):
a. Tₘ=69.3+0.41(G+C)%
b. The Tₘ of a duplex DNA decreases by 1° C. with every increase of 1% in the number of mismatched base pairs.
c. (Tₘ)_{μ2}−(Tₘ)_{μ1}=18.5 log₁₀ μ2/μ1
where μ1 and μ2 are the ionic strengths of two solutions.

As is well known in the art, hybridization stringency is a function of many factors, including overall DNA concentration, ionic strength, temperature, probe size and the presence of agents which disrupt hydrogen bonding. Factors promoting hybridization include high DNA concentrations, high ionic strengths, low temperatures, longer probe size and the absence of agents that disrupt hydrogen bonding. Hybridization typically is performed in two phases: the “binding” phase and the “washing” phase.

### Functionally Equivalent Variants

Yet another class of nucleic acids within the scope of the disclosure may be described with reference to the product they encode. These functionally equivalent polynucleotides are characterized by the fact that they encode the same peptide sequences, for example those found in SEQ ID NOs: 15-25, due to the degeneracy of the genetic code. In some embodiments, the polynucleotides of the disclosure are functionally equivalent polynucleotides.

It is recognized that variants of the nucleic acids provided herein can be constructed in several different ways. In some embodiments the polynucleotides are comprised of synthetic nucleic acids (e.g. synthetic DNAs). Methods of efficiently synthesizing oligonucleotides in the range of 20 to about 150 nucleotides are widely available. See Ausubel et al., section 2.11, Supplement 21 (1993). Overlapping oligonucleotides may be synthesized and assembled in a fashion first reported by Khorana et al., J. Mol. Biol. 72:209-217 (1971); see also Ausubel et al., supra, Section 8.2. In some embodiments, Synthetic nucleic acids are designed with convenient restriction sites engineered at the 5′ and 3′ ends of the gene to facilitate cloning into an appropriate vector.

In some embodiments, a method of generating variants is to start with one of the nucleic acids disclosed herein and then to conduct site-directed mutagenesis. See Ausubel et al., supra, chapter 8, Supplement 37 (1997). In some embodiments, a target nucleic acid, e.g., DNA, is cloned into a single-stranded DNA bacteriophage vehicle. Single-stranded DNA is isolated and hybridized with an oligonucleotide containing the desired nucleotide alteration(s). The complementary strand is synthesized and the double stranded phage is introduced into a host. Some of the resulting progeny will contain the desired mutant, which can be confirmed using DNA sequencing. In addition, various methods are available that increase the probability that the progeny phage will be the desired mutant. These and other methods of gene or DNA synthesis and modification are well known to those in the field and kits are commercially available for generating such mutants.

### Recombinant Nucleic Acid Constructs and Expression

The present disclosure further provides recombinant nucleic acid constructs comprising one or more of the nucleotide sequences of the present disclosure. The recombinant constructs of the present disclosure are used in connection with a vector, such as a plasmid, phagemid, phage or viral vector, into which a nucleic acid molecule encoding a PDGFRbeta-Fc fusion protein of the disclosure is inserted.

In some embodiments, a PDGFRbeta-Fc fusion protein or derivative thereof provided herein can be prepared by recombinant expression of nucleic acid sequences encoding a PDGFRbeta-Fc fusion protein or portions thereof in a host cell. In some embodiments, to express a PDGFRbeta-Fc fusion protein or derivative thereof recombinantly, a host cell can be transfected with one or more recombinant expression vectors carrying nucleic acid fragments encoding a PDGFRbeta-Fc fusion protein or portions thereof such that the PDGFRbeta-Fc fusion protein is expressed in the host cell. Standard recombinant nucleic acid methodologies may be used to prepare and/or obtain nucleic acids encoding the PDGFRbeta-Fc fusion protein, incorporate these nucleic acids into recombinant expression vectors and introduce the vectors into host cells, such as those described in Sambrook, Fritsch and Maniatis (eds.), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

In some embodiments, to express the PDGFRbeta-Fc fusion proteins or derivatives thereof, standard recombinant nucleic acid expression methods can be used (see, for example, Goeddel; Gene Expression Technology. Methods in Enzymology 185, Academic Press, San Diego, Calif. (1990)). For example, a nucleic acid encoding the desired polypeptide can be inserted into an expression vector which is then transfected into a suitable host cell. Suitable host cells are prokaryotic and eukaryotic cells. Examples for prokaryotic host cells are e.g. bacteria, examples for eukaryotic host cells are yeast, insect, plant or mammalian cells. It is understood that the design of the expression vector, including the selection of regulatory sequences is affected by factors such as the choice of the host cell, the level of expression of protein desired and whether expression is constitutive or inducible.

### Bacterial Expression

In some embodiments, useful expression vectors for bacterial use are constructed by inserting a structural nucleic acid (e.g. DNA) sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus.

In some embodiments, bacterial vectors may be, for example, bacteriophage-, plasmid- or phagemid-based. These vectors can contain a selectable marker and bacterial origin of replication derived from commercially available plasmids typically containing elements of the well-known cloning vector pBR322 (ATCC 37017). Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification. Alternatively, where the desired protein is secreted from the cells, cells are typically harvested by centrifugation, discarded and the resulting supernatants are retained for further purification. In bacterial systems, a number of expression vectors may be advantageously selected depending upon the use intended for the protein being expressed. For example, when a large quantity of such a protein is to be produced, vectors which direct the expression to high levels of fusion protein products, that are readily purified, may be desirable.

PDGFRbeta-Fc fusion proteins of the present disclosure include products of chemical synthetic procedures, and products produced by recombinant techniques from a prokaryotic host, including, for example, E. coli, Bacillus subtilis, Salmonella typhimurium and various species within the genera Pseudomonas, Streptomyces, and Staphylococcus.

### Mammalian Expression & Purification

In some embodiments, regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al. In some embodiments, the recombinant expression vectors can also include origins of replication and selectable markers (see e.g., U.S. Pat. Nos. 4,399,216, 4,634,665 and U.S. Pat. No. 5,179,017, by Axel et al.). In some embodiments, suitable selectable markers include genes that confer resistance to drugs such as G418, hygromycin or methotrexate, on a host cell into which the vector has been introduced. For example, the dihydrofolate reductase (DHFR) gene confers resistance to methotrexate and the neo gene confers resistance to G418. In some embodiments, transfection of the expression vector into a host cell can be carried out using standard techniques such as electroporation, calcium-phosphate precipitation, and DEAE-dextran transfection.

Therefore, an embodiment of the present disclosure is an expression vector comprising a nucleic acid sequence encoding for the PDGFRbeta-Fc fusion proteins of the present disclosure. See Example 1 for an exemplary description.

Suitable mammalian host cells for expressing the PDGFRbeta-Fc fusion proteins, or derivatives thereof provided herein include Chinese Hamster Ovary (CHO cells) [including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621], NSO myeloma cells, COS cells and SP2 cells. In some embodiments, the expression vector is designed such that the expressed protein is secreted into the culture medium in which the host cells are grown. The PDGFRbeta-Fc fusion proteins or derivatives thereof can be recovered from the culture medium using standard protein purification methods.

PDGFRbeta-Fc fusion proteins of the disclosure can be recovered and purified from recombinant cell cultures by well-known methods including, but not limited to ammonium sulfate or ethanol precipitation, acid extraction, Protein A chromatography, Protein G chromatography, anion or cation exchange chromatography, phospho-cellulose chromatography, hydrophobic interaction chromatography, affinity chromatography, hydroxylapatite chromatography, size exclusion chromatography, reversed phase chromatography and lectin chromatography. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

In some embodiments, the PDGFRbeta-Fc fusion proteins of the present disclosure include naturally purified products, products of chemical synthetic procedures, and products produced by recombinant techniques from a eukaryotic host, including, for example, yeast, plant, insect and mammalian cells. Depending upon the host employed in a recombinant production procedure, the PDGFRbeta-Fc fusion proteins of the present disclosure can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20.

Therefore, an embodiment of the present disclosure are also host cells comprising the vector or a nucleic acid molecule, whereby the host cell can be a higher eukaryotic host cell, such as a mammalian cell, a lower eukaryotic host cell, such as a yeast cell, and may be a prokaryotic cell, such as a bacterial cell.

Another embodiment of the present disclosure is a method of using the host cell to produce a PDGFRbeta-Fc fusion protein, comprising culturing the host cell under suitable conditions and recovering said PDGFRbeta-Fc fusion protein.

### Therapeutic Methods

In some embodiments, therapeutic methods involve administering to a subject in need of treatment a therapeutically effective amount of a PDGFRbeta-Fc fusion protein contemplated by the disclosure. A “therapeutically effective” amount hereby is defined as the amount of an PDGFRbeta-Fc fusion protein that is of sufficient quantity to deplete PDGF ligands and abrogate PDGF signaling in a treated area of a subject—either as a single dose or according to a multiple dose regimen, alone or in combination with other agents, which leads to the alleviation of an adverse condition, yet which amount is toxicologically tolerable. The subject may be a human or non-human animal (e.g., rabbit, rat, mouse, dog, monkey or other lower-order primate).

In some embodiments, the PDGFR-Fc fusion proteins of the present disclosure are co-administered with known medicaments, and in some embodiments the fusion protein might itself be modified. PDGFRbeta-Fc fusion proteins of the present disclosure may be administered as the sole pharmaceutical agent or in combination with one or more additional therapeutic agents where the combination causes no unacceptable adverse effects. In some embodiments, this combination therapy includes administration of a single pharmaceutical dosage formulation which contains PDGFRbeta-Fc fusion protein of the disclosure and one or more additional therapeutic agents, as well as administration of a PDGFRbeta-Fc fusion protein of the disclosure and each additional therapeutic agent in its own separate pharmaceutical dosage formulation. In some embodiments, PDGFRbeta-Fc fusion proteins of the disclosure and a therapeutic agent may be administered to the patient together in a single dosage composition, or each agent may be administered in separate dosage formulations.

Where separate dosage formulations are used, a PDGFRbeta-Fc fusion protein of the disclosure and one or more additional therapeutic agents may be administered at essentially the same time (e.g., concurrently) or at separately staggered times (e.g., sequentially).

### Pharmaceutical Compositions and Administration

The present disclosure also provides pharmaceutical compositions which comprise a PDGFR-Fc fusion protein, alone or in combination with at least one other agent, such as stabilizing compound, which may be administered in any sterile, biocompatible pharmaceutical carrier, including, but not limited to, saline, buffered saline, dextrose, and water.

In some embodiments, the pharmaceutical compositions of the disclosure may also comprise, in addition to a PDGFRbeta-Fc fusion protein, a pharmaceutically active compound that is suitable to treat PDGFRbeta related diseases such as cancer, back-of-the-eye diseases such as age-related macular degeneration (AMD), choroidal neovascularization (CNV), choroidal neovascular membrane (CNVM), cystoid macula edema (CME), epi-retinal membrane (ERM) and macular hole, myopia-associated choroidal neovascularisation, vascular streaks, retinal detachment, diabetic retinopathy, diabetic macular edema (DME), atrophic changes of the retinal pigment epithelium (RPE), hypertrophic changes of the retinal pigment epithelium (RPE), retinal vein occlusion, choroidal retinal vein occlusion, macular edema, macular edema due to retinal vein occlusion, proliferative vitreoretinopathy (PVR), familiar exudative vitreoretionpathy (FEVR), retinitis pigmentosa, Stargardt's disease and retinopathy of prematurity. In addition, examples include but are not limited to angiogenesis in the front of the eye like corneal angiogenesis following e.g. keratitis, corneal transplantation or keratoplasty, corneal angiogenesis due to hypoxia (extensive contact lens wearing), pterygium conjunctivae, subretinal edema and intraretinal edema. In some embodiments, the described PDGFRbeta-Fc fusion proteins are useful for the treatment of nephropathies such as diabetic nephropathy and fibrotic disease such as pulmonary fibrosis. Any of these molecules can be administered to a patient alone, or in combination with other agents, drugs or hormones, in pharmaceutical compositions where it is mixed with excipient(s) or pharmaceutically acceptable carriers.

In some embodiments, the present disclosure also relates to the administration of pharmaceutical compositions. Such administration may be accomplished orally or parenterally. Methods of parenteral delivery include but are not limited to intravitreal, topical, intra-arterial (directly to the tumor), intramuscular, subcutaneous, intramedullary, intrathecal, intraventricular, intravenous, intraperitoneal, or intranasal administration. In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Ed. Maack Publishing Co, Easton, Pa.). In a particular embodiment, the application of the PDGFRbeta-Fc fusion protein to the eye is performed by intravitreal injection.

In some embodiments, pharmaceutical compositions for administration can be formulated using pharmaceutically acceptable carriers well known in the art in dosages suitable for oral administration. Such carriers enable the pharmaceutical compositions to be formulated as e.g. tablets, pills, dragees, capsules, liquids, creams, gels including hydrogels, ointments, syrups, slurries, suspensions, sprays, aerosols, injectables, implants and the like. Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl, cellulose, hydroxypropylmethylcellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate. In some embodiments, the pharmaceutical compositions are substantially pyrogen free. In some embodiments, the pharmaceutical compositions are pyrogen free.

Dragee cores are provided with suitable coatings such as concentrated sugar solutions, which may also contain gum arabic, talc, polyvinyl pyrrolidone, carbopol gel, polyethylene glycol and/or titanium dioxide, lacquer solutions, and suitable organic solvents or solvent mixtures. Dyestuffs or pigments may be added to the tablets or dragee coatings for product identification or to characterize the quantity of active compound, i.e. dosage.

Pharmaceutical preparations that can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous or non-aqueous solutions, suspensions, or emulsions of active compounds. For injection, the pharmaceutical compositions of the disclosure may be formulated in aqueous solutions. In some embodiments, the compositions are formulated in physiologically compatible buffers such as Hank's solution, Ringer's solution, or physiologically buffered saline e.g. phosphate buffered saline. Aqueous injection suspensions may contain substances that increase viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

An aqueous formulation for parenteral administration may contain 1-200 mg/ml of the PDGFRbeta-Fc fusion protein according to the disclosure. In some embodiments, a formulation for parenteral administration contains 1-200 mg/mL, 1-100 mg/ml, 5-50 mg/ml, 1-50 mg/ml or 1-25 mg/ml mg/ml of the PDGFRbeta-Fc fusion protein according to the disclosure. In yet other embodiments, a formulation for parenteral administration contains 8-30 mg/ml of the PDGFRbeta-Fc fusion protein according to the disclosure.

For topical or nasal administration, penetrants appropriate to the particular barrier to be permeated are used in the formulation. Such penetrants are generally known in the art.

### Kits

The disclosure further relates to pharmaceutical packs and kits comprising one or more containers filled with one or more of the ingredients of the aforementioned compositions of the disclosure. Associated with such container(s) can be a notice in the form prescribed by a governmental agency regulating the manufacture, use or sale of pharmaceuticals or biological products, reflecting approval by the agency of the manufacture, use or sale of the product for human administration.

In another embodiment, the kits may contain polynucleotide sequences encoding the PDGFRbeta-Fc fusion proteins of the disclosure. In some embodiments, the polynucleotide is RNA. In some embodiments, the polynucleotide is DNA. In some embodiments, the polynucleotide sequences encoding these fusion proteins are provided in a plasmid suitable for transfection into and expression by a host cell. The plasmid may contain a promoter (often an inducible promoter) to regulate expression of the polynucleotide in the host cell. The plasmid may also contain appropriate restriction sites to facilitate the insertion of other polynucleotide sequences into the plasmid to produce various proteins. The plasmids may also contain numerous other elements to facilitate cloning and expression of the encoded proteins. Such elements are well known to those of skill in the art and include, for example, selectable markers, initiation codons, termination codons, and the like.

### Manufacture and Storage.

The pharmaceutical compositions of the present disclosure may be manufactured in a manner that is known in the art, e.g., by means of conventional mixing, dissolving, granulating, dragee-making, levigating, emulsifying, encapsulating, entrapping or lyophilizing processes.

In some embodiments, the pharmaceutical composition may be provided as a salt and can be formed with acids, including by not limited to hydrochloric, sulfuric, acetic, lactic, tartaric, malic, succinic, etc. Salts tend to be more soluble in aqueous or other protonic solvents than are the corresponding free base forms. This composition may be further enhanced by inclusion of sugars, such as but not limited to sucrose, trehalose, mannitol or glucose. In some embodiments, the composition may further contain other excipients like detergents, polyols or substances used in protein or antibody formulations by the skilled in the art. In other cases, the preparation may be a lyophilized powder that is combined with buffer prior to use.

After pharmaceutical compositions comprising a compound of the disclosure formulated in an acceptable carrier have been prepared, they can be placed in an appropriate container and labeled for treatment of an indicated condition. For administration of PDGFRbeta-Fc fusion proteins, such labeling would include amount, frequency and method of administration.

### Therapeutically Effective Dose.

Pharmaceutical compositions suitable for use in the present disclosure include compositions wherein the active ingredients are contained in an effective amount to achieve the intended purpose, i.e. treatment of a particular disease state characterized by PDGFRbeta expression. The determination of an effective dose is well within the capability of those skilled in the art.

For any compound, the therapeutically effective dose can be estimated initially either in cell culture assays, e.g. using neoplastic cells, primary or immortalized fibroblasts or endothelial cells, or in animal models, usually mice, rats, rabbits, dogs, pigs or monkeys. The animal model is also used to achieve a desirable concentration range and route of administration. Such information can then be used to determine useful doses and routes for administration in humans.

A therapeutically effective dose refers to that amount of PDGFRbeta fusion protein or biologically active fragment thereof, that ameliorate the symptoms or condition. Therapeutic efficacy and toxicity of such compounds can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. In some embodiments, the pharmaceutical compositions exhibit large therapeutic indices. The data obtained from cell culture assays and animal studies are used in formulating a range of dosage for human use. In some embodiments, the dosage of such compounds lies within a range of circulating concentrations what include the ED₅₀ with little or no toxicity. The dosage varies within this range depending upon the dosage form employed, sensitivity of the patient, and the route of administration.

The exact dosage of PDGFRbeta-Fc fusion protein or biologically active fragment thereof to be administered to a subject is chosen by the individual physician in view of the patient to be treated. Dosage and administration are adjusted to provide sufficient levels of the active moiety or to maintain the desired effect. Additional factors that may be taken into account include the severity of the disease state, e.g., tumor size and location; change in visual acuity, retinal thickness determined e.g. by Optical Coherence Tomography (OCT), functional retinal changes monitored e.g. in Electroretinograms (ER); age, weight and gender of the patient; diet, time and frequency of administration, drug combination(s), reaction sensitivities, and tolerance/response to therapy. In some embodiments, long acting pharmaceutical compositions might be administered every day, or every 2 days, or every 3 to 4 days, or every week, or once every two weeks, or once every month, or once every two months, or once every quarter of the year, or once every half of the year, or every year depending on half-life and clearance rate of the particular formulation.

Normal dosage amounts may vary from 0.1 to 100,000 micrograms, up to a total dose of about 2 g, depending upon the route of administration and the factors mentioned above. Guidance as to particular dosages and methods of delivery is provided in the literature. See U.S. Pat. Nos. 4,657,760; 5,206,344; or 5,225,212. Those skilled in the art will employ different formulations for polynucleotides than for proteins or their inhibitors. Similarly, delivery of polynucleotides or polypeptides will be specific to particular cells, conditions, locations, etc.

When ophthalmologically administered to a human, the PDGFbeta-Fc fusion protein, or a biologically active fragment thereof, according to the disclosure is injected into the eye in volumes of about 5-150 μl, of about 25-125 μl, or about 10-50 μl, wherein the concentration of the protein is about 1-200 mg/ml, about 5-50 mg/ml, or about 8-30 mg/ml.

### Method of Treating Ophthalmological Disorders

The present disclosure also relates to a use of the pharmaceutical composition according to the disclosure to treat or prevent ophthalmological disorders.

Furthermore, the present disclosure also relates to a method for treating or preventing an ophthalmological disorder comprising administering to a subject a pharmaceutical composition containing a pharmaceutically effective amount of a PDGFRbeta-Fc fusion according to the present disclosure. In some embodiments, the administration of a PDGFRbeta-Fc fusion protein described herein decreases neovascularization. In some embodiments, the administration of a PDGFRbeta-Fc fusion protein decreases neovascularization by at least 5%, or at least 10%, or at least 15%, or at least 20%, or at least 25%, or at least 30%, or at least 40%, or at least 50% compared to an untreated a control subject. In further embodiments, the administration does not substantially increase vascular leakage, where herein “does not substantially increase vascular leakage” means that the vascular leakage score determined by angiography increases by no more than 15%, 10%, 8%, 5%, 2% or 1% as compared to vascular leakage observed in an untreated control subject. In some embodiments, the administration decreases vascular leakage as compared to the vascular leakage observed in an untreated control subject. In some embodiments, the administration decreases vascular leakage by at least 10%, 20%, 30%, 40%, 50%, 60%, 70%, 80%, 90% or 100% as compared to the vascular leakage observed in an untreated control subject.

In a further aspect the disclosure provides a method for inhibiting ocular neovascularization, comprising administering to a subject a PDGFRbeta-Fc fusion protein described herein, wherein said administration reduces vascular leakage. In some embodiments, the administration decreases vascular leakage by at least 10%, 20%, 30%, 40%, 50%, 60%, 70%, 80%, 90% or 100% as compared to the vascular leakage observed in an untreated control subject. In a further embodiment, the administration decreases neovascularization. In some embodiments the administration of a PDGFRbeta-Fc fusion protein decreases neovascularization by at least 5%, or at least 10%, or at least 15%, or at least 20%, or at least 25%, or at least 30%, or at least 40%, or at least 50% compared to an untreated control subject.

Examples of ophthalmological disorders according to the disclosure include, but are not limited to retinal disorders, macular degeneration such as age-related macular degeneration (AMD), non-exudative and exudative age-related macular degeneration, choroidal neovascularization (CNV), choroidal neovascular membrane (CNVM), macular edema such as cystoid macular edema (CME) or diabetic macular edema (DME), epi-retinal membrane disorders (ERM), macular hole, myopia-associated choroidal neovascularisation, vascular streaks, retinal detachment, diabetic retinopathy, atrophic changes of the retinal pigment epithelium (RPE), hypertrophic changes of the retinal pigment epithelium (RPE), retinal arterial occlusive diseases, retinal vein occlusion such as central retinal vein occlusion (CVRO), branch retinal vein occlusion (BRVO), choroidal retinal vein occlusion, macular edema, macular edema due to retinal vein occlusion, retinitis pigmentosa, Stargardt's disease, glaucoma, inflammatory conditions of the eye such as uveitis, scleritis or endophthalmitis, cataract, refractory anomalies such as myopia, hyperopia or astigmatism, ceratoconus, acute or chronic macular neuroretinopathy, Behcet's disease, ocular trauma effecting a posterior ocular site or location, posterioar ocular condition caused by or influenced by an ocular laser treatment or by photodynamic therapy, photocoagulation, radiation retinopathy, anterior ischemic optic neuropathy, non-retinopathy diabetic retinal disfunction, retinitis, pigmentosa, central serious choroiretinopathy, choroiditis, acute multifocal placoid pigment epitheliopathy, birdshot retinochoroiopathy, multiple evanescent white dot syndrome, ocular sarcoidosis, posterior scleritis, serpignous choroiditis, subretinal fibrosis, Vogt-Koyanagi-Harada syndrome, disseminated intravascular coagulopathy, ocular ischemic syndrome, retinal arterial microaneurysms, Coat's disease, parafoveal telangiectasis, carotid artery disease, frosted branch angitis, hemoglobolinopathies such as sickle cell retinopathy, angioid streaks, exudative vitreoretinopathy, Eales disease, traumatic eye disease e.g. after eye surgery such as sympathetic ophtalmia, uveitic retinal disease, retinal detachment, photocoagulation or bone marrow transplant retinopathy, ocular histoplasmosis, ocular toxocariasis, viral retinitis, acute rtinal necrosis, progressive outer retinal necrosis, fungal retinal diseases, ocular syphilis, ocular tuberculosis, diffuse unilateral subcacute neuroretinitis, retinal dystrophies, congenital stationary night blindness, cone dystrophies, fundus flavimaculatus, Bests disease, pattern dystrophy of retinal pigmented epithelium, X-linked retinoschisis, Sorsby funduns dystrophy, benign concentric maculopathy, Biett's crystalline dystrophy, pseudoxanthoma elasticum, congenital hypertrophy of RPE, posterior uveal melanoma, choroidal meangioma, choroidal osteoma, choroidal metastatis, combined hamartoma of the retina and retinal pigmented epithelium, retinoblastoma, vasoproliferative tumors of the ocular fundus, retinal astrocytoma, intraocular lymphoid tumors, punctate inner choroidopathy, acute posterior multifocal placid pigment epitheliopathy, myopic retinal degeneration, acute retinal pigment epithelitis and retinopathy of prematurity. In addition, examples include but are not limited to angiogenesis in the front of the eye like corneal angiogenesis following e.g. keratitis, corneal transplantation or keratoplasty, corneal angiogenesis due to hypoxia (extensive contact lens wearing), pterygium conjunctivae, subretinal edema and intraretinal edema. Examples of age-related macular degeneration (AMD) include but are not limited to dry or nonexudative AMD, or wet or exudative or neovascular AMD.

In some embodiments, the method comprises administering a pharmaceutical composition containing a pharmaceutically effective amount of a PDGFRbeta-Fc fusion according to the present disclosure to treat age-related macular degeneration (AMD) such as dry AMD or wet AMD, choroidal neovascularization (CNV), choroidal neovascular membrane (CNVM), macular edema such as cystoid macular edema (CME) or diabetic macular edema (DME). In some embodiments, the method comprises treating AMD.

The pharmaceutical composition according to the disclosure can be administered as the sole pharmaceutical composition or in combination with one or more other pharmaceutical compositions or active agents where the combination causes no unacceptable adverse effects.

“Combination” means for the purposes of the disclosure not only a dosage form which contains all the active agents (so-called fixed combinations), and combination packs containing the active agents separate from one another, but also active agents which are administered simultaneously or sequentially, as long as they are employed for the prophylaxis or treatment of the same disease.

Since the combination according to the disclosure is well tolerated in an animal model and is potentially effective even in low dosages, a wide range of formulation approaches is possible. Thus, one possibility is to formulate the individual active ingredients of the combination according to the disclosure either as a solution or mixture, as in a “single syringe” device, or separately. In the latter case, it is not absolutely necessary for the individual active ingredients to be taken at the same time; on the contrary, sequential intake may be advantageous to achieve optimal effects. In some embodiments, it is appropriate with such separate administration to combine the formulations of the individual active ingredients simultaneously together in a suitable primary packaging, or in a “device” that facilitates administration. The active ingredients are then present in the primary packaging or device in one or separate containers which may be, for example, syringes, double-chamber syringes, tubes, bottles or blister packs. In some embodiments, separate packaging of the components in the joint primary packaging is also referred to as a kit.

In one embodiment, the pharmaceutical compositions of the present disclosure can be combined with other ophthalmological agents. Examples of such agents include, but are not limited to carotenoids like lycopene, lutein, zeaxanthin, phytoene, phytofluene, carnosic acid and derivatives thereof like carnosol, 6,7-dehydrocarnosic acid, 7-ketocarnosic acid, a zink source like zinc oxide or a zinc salt like its chloride, acetate, gluconate, carbonate, sulphate, borate, nitrate or silicate salt, copper oxide, vitamin A, vitamin C, vitamin E and/or β-carotene.

In another embodiment, the pharmaceutical compositions of the present disclosure can be combined with other signal transduction inhibitors targeting receptor kinases of e.g. VEGFR, IGFR, FGFR, HGFR and their respective ligands or other pathway inhibitors like VEGF-Trap (aflibercept), FGFR fusion proteins, regorafenib, pegaptanib, ranibizumab, pazopanib, bevasiranib, KH-902, mecamylamine, PF-04523655, E-10030, ACU-4429, volociximab, sirolismus, fenretinide, disulfiram, sonepcizumab and/or tandospirone. These agents include, by no way of limitation, antibodies such as Avastin (bevacizumab). These agents also include, by no way of limitation, small-molecule inhibitors such as STI-571/Gleevec (Novartis), PTK-787 (Wood et al., Cancer Res. 2000, 60(8), 2178-2189), SU-11248 (Demetri et al., Proceedings of the American Society for Clinical Oncology 2004, 23, abstract 3001), ZD-6474 (Hennequin et al., 92nd AACR Meeting, New Orleans, Mar. 24-28, 2001, abstract 3152), AG-13736 (Herbst et al., Clin. Cancer Res. 2003, 9, 16 (suppl 1), abstract C253), KRN-951 (Taguchi et al., 95th AACR Meeting, Orlando, Fla., 2004, abstract 2575), CP-547,632 (Beebe et al., Cancer Res. 2003, 63, 7301-7309), CP-673,451 (Roberts et al., Proceedings of the American Association of Cancer Research 2004, 45, abstract 3989), CHIR-258 (Lee et al., Proceedings of the American Association of Cancer Research 2004, 45, abstract 2130), MLN-518 (Shen et al., Blood 2003, 102, 11, abstract 476), and AZD-2171 (Hennequin et al., Proceedings of the American Association of Cancer Research 2004, 45, abstract 4539), PKC412, nepafenac.

In some embodiments, the PDGFRbeta-Fc fusion proteins of the present disclosure are administered to a subject in combination with a receptor tyrosine kinase antagonist. In certain embodiments of the present invention, the receptor tyrosine kinase antagonist with which the PDGFRbeta-Fc fusion protein of the present invention can be combined is a VEGFR antagonist, IGFR antagonist, FGFR antagonist or a HGFR antagonist. In some embodiments, the PDGFRbeta-Fc fusion protein (or biologically active fragment thereof) and the receptor tyrosine kinase signaling antagonist are administered simultaneously or within 90 days of each other. In a further embodiment, subsequent administration of PDGFRbeta-Fc fusion protein is given biweekly, monthly or bimonthly for 1, 2, 3, 4, 5, 6, 7, 8 9, 10, 11 or 12 months after the initial treatment, or continued for years up to the lifetime of the patient. In some embodiments, the PDGFRbeta-Fc fusion protein (or biologically active fragment thereof) and receptor tyrosine kinase signaling antagonists are administered in parallel, wherein the PDGFRbeta-Fc fusion protein or biologically active fragment thereof is administered at biweekly, monthly or bimonthly intervals for 1, 2, 3, 4, 5, 6, 7, 8, 9, 10, 11 or 12 months, or for years, and receptor tyrosine kinase antagonist is administered monthly, or biweekly, or weekly, or daily, or up to 5 times per day for 1 day, or 1 week, or 2 weeks, or 1 month, or 2 months, or 3 months, or 6 months, or 9 months, or 12 months, or for years. In some embodiments, the PDGFRbeta-Fc or biologically active fragment thereof and receptor tyrosine kinase signaling antagonists are administered in parallel, wherein the PDGFRbeta-Fc (or biologically active fragment thereof) is administered at biweekly, monthly or bimonthly intervals for 1, 2, 3, 4, 5, 6, 7, 8, 9, 10, 11, 12 months, or years, and receptor tyrosine kinase agonist is administered once. In some embodiments the receptor tyrosine kinase signaling antagonist is a sustained release formulation.

In some embodiments, the pharmaceutical compositions of the present disclosure is combined with VEGF-Trap (aflibercept), regorafenib, FGFR fusion proteins, bevacizumab, pegaptanib, ranibizumab, pazopanib and/or bevasiranib. In some embodiments, combinations include combination c-met fusion proteins or antibodies against c-met or HGF, antibodies or fusion proteins against IGFR-I and/or II, IGF-I and or -II antibodies, and/or FGFs or FGFRs, Integrins, Cadherins, Cathepsins, MMPs, ADAMs, Nicotinic receptors, vascular disrupting agents, proteins of the complement system, steroids, chemokine/cytokine antibodies or chemokine/cytokine receptor antagonists like e.g. but not limited to agents targeting MCP-1/CCL2, Eotaxin-1/CCL, Eotaxin-2/CCL, Eotaxin-3/CCL, SDF-1alpha/CXCL12, CCR2, CCR3, CXCR4.

In further embodiments, the receptor tyrosine kinase antagonist is a VEGF antagonist. In some embodiments, the VEGF antagonist is a small molecule, an antibody, a VEGF trap, an aptamer, a RNAi construct or an antisense construct. In still further embodiments, the VEGF antagonist is regorafenib, a hydrate, solvate or pharmaceutical acceptable salt thereof or a polymorph thereof. In a further embodiment, the regorafenib is administered in a sustained release formulation.

A sustained release formulation is a formulation that on a single administration will continue to provide or release an active agent over an extended period of time. In some embodiments, a sustained release composition is an implant which may be solid, semisolid or viscoelastic. In some embodiments, the active agent may be released from the implant by diffusion, erosion or degradation of the polymer matrix, dissolution or osmosis of the active agent or swelling of the polymer matrix. In some embodiments, the polymer matrix agents are pharmaceutically acceptable and biocompatible with the eye and can be biodegradable or non-biodegradable. In some embodiments, the polymer matrix agents are biodegradable. The implant comprising a biodegradable polymer matrix agent may partially or completely disappear in the eye by degradation or erosion which can be e.g. enzymatically or hydrolytically. In some embodiments, the implant may completely disappear in the eye by degradation or erosion. The polymer matrix agent can be cross-linked or non-cross-linked. In some embodiments, the polymer matrix agent is biocompatible with the eye and does not cause any substantial interference with the functioning or physiology of the eye. Examples of polymer matrix agents include but are not limited to polymers of hydroxyaliphatic carboxylic acids, polysaccharides (e.g. alginates, cellulose and derivatives thereof like carboxymethylcellulose and esters thereof), polymers of lactic acid (either in the D- or L-form or as a racemic mixture) such as polylactides (PLA), polymers of glycolic acid such as polyglycolides (PGA), poly-lactide-co-glycolide (PLGA), polycaprolactone, polyesters, poly(ortho esters), poly(phosphazine), poly(phosphate ester), natural polymers such as gelatin or collagen, or mixtures of the before mentioned agents. In some embodiments, the sustained release composition provides a sustained, controlled and/or extended delivery of the active agent at a maintained level of 0.01 μg to 100 μg per day, 0.01 μg to 50 μg per day, or 0.1 μg to 25 μg per day, or 0.5 μg to 15 μg per day, or 1 μg to 10 μg per day. In some embodiments, it releases the active agent in a sustained manner for a period of time of 3 months, or 4 months, or 5 months, or 6 months, or 8 months, or 10 months, or 12 months, or 14 months or 16 months or 18 months after the implant is placed into the eye.

In cases in which a combination with a second injectable or implantable ophthalmological drug which is administered intravitreally to the eye by injection of a solution or suspension or by implantation of a sustained release composition, the injection or implantation may be done in parallel and at essentially the same time point with the present injection or sequentially. In an embodiment, the administration (e.g. injection) of the ophthalmological drug according to the present disclosure can be repeated in the first month, or 2 months, or 3 months, or 4 months, or 5 months, or 6 months after implantation of a sustained release composition containing a second ophthalmological drug on a monthly basis.

In some embodiments, the PDGFRbeta-Fc fusion proteins of the present disclosure can be combined with an eye drop composition containing a second ophthalmological drug, e.g. but not limited to an inhibitor of the VEGF pathway, which can be topically delivered into the eye. These eye drops can be administered one or more times per day, up to 5 times per day, or up to 3 times per day. For example, the injection of the present ophthalmological drug can be done each 1, or 2, or 3, or 4, or 5, or up to 6 months in parallel with the treatment with the eye drops containing a second ophthalmological drug administered up to 5 times a day. In some embodiments, the eye drop composition contains regorafenib, a hydrate, solvate or pharmaceutical acceptable salt thereof or a polymorph thereof as particularly described in WO 2013/000917.

Generally, the use of the other ophthalmological agents in combination with the pharmaceutical compositions of the present disclosure will serve to:
(1) yield better efficacy as compared to administration of either agent alone,
(2) provide for the administration of lesser amounts or fewer applications of the administered agents,
(3) provide for treating a broader spectrum of mammals, especially humans,
(4) provide for a higher response rate among treated patients,
(5) yield efficacy and tolerability results at least as good as those of the agents used alone, compared to known instances where other agent combinations produce antagonistic effects. It is believed that one skilled in the art, using the preceding information and information available in the art, can utilize the present disclosure to its fullest extent.

In particular, the use of an inhibitor of the VEGF pathway in combination with a PDGFRbeta-Fc fusion protein according to the present disclosure has the advantage to not increase vascular leakage in contrast to other known compounds targeting PDGF signaling in angiogenesis (see Example 4.2.2, FIG. 6B).

It should be apparent to one of ordinary skill in the art that changes and modifications can be made to this disclosure without departing from the spirit or scope of the disclosure as it is set forth herein.

All publications, applications and patents cited above and below are incorporated herein by reference.

The weight data are, unless stated otherwise, percentages by weight and parts are parts by weight.

The present disclosure is further described by the following examples. The examples are provided solely to illustrate the disclosure by reference to specific embodiments. These exemplifications, while illustrating certain specific aspects of the disclosure, do not portray the limitations or circumscribe the scope of the disclosure.

All examples were carried out using standard techniques, which are well known and routine to those of skill in the art, except where otherwise described in detail. Routine molecular biology techniques of the following examples can be carried out as described in standard laboratory manuals, such as Sambrook et al., Molecular Cloning: A Laboratory Manual, 2nd Ed.; Cold Spring Harbor Laboratory Press, Cold Spring Harbor, N. Y., 1989.

### EXAMPLES

### Example 1

### Generation of Different Recombinant PDGFRbeta Fc Constructs

To generate different Fc fusion proteins (SEQ ID NO: 15-25) the extracellular domains 1-3 of PDGFRbeta, derived from UNIPROT ID P09619 (SEQ ID NO: 2 and FIG. 1A) were C-terminally fused to a human IgG1 Fc-part (SEQ ID NO: 14). This was done either directly (SEQ ID NO: 15) or via different amino acid linkers (SEQ ID NO: 16-25 see FIG. 1 B). The linker was a GGGGS linker selected from SEQ ID NO: 7-13, i.e.

SEQ ID NO 7:
GGGGS (1x GGGGS),
SEQ ID NO 8:
GGGGSGGGGS (2x GGGGS),
SEQ ID NO 9:
GGGGSGGGGSGGGGS (3x GGGGS),
SEQ ID NO 10:
GGGGSGGGGSGGGGSGGGGS (4x GGGGS),
SEQ ID NO 11:
GGGGSGGGGSGGGGSGGGGSGGGGS (5x GGGGS),
SEQ ID NO 12:
GGGGSGGGGSGGGGSGGGGSGGGGSGGGGS (6x GGGGS),
SEQ ID NO 13:
GGGGSGGGGSGGGGSGGGGSGGGGSGGGGSGGGGS (7x GGGGS),

or a linker selected from SEQ ID NOs: 4-6, i.e SEQ ID NO 4: EPKSC, SEQ ID NO 5: EPKSS or SEQ ID NO 6: GGGGG. The gene of the respective Fc fusion protein was cloned into a suitable expression vector based on a CMV promoter system for protein expression in mammalian cells. HEK293 6E cells were transiently transfected and the cell culture scale was up to 1.5 L in a shake flask or 10 L in a Cultibag fermenter (Sartorius). Expression was done at 37° C. for 5-days. As medium F17 (LifeTechnologies) supplemented with Tryptone TN1 (Organotechnie), 1% “FCS ultra low IgG” (Invitrogen) and 0.5 mM valproic acid was used.

The PDGFRbeta-Fc fusion proteins, e.g. SED IDs 15-25, were purified from mammalian cell supernatants. Cells were removed by a suitable technique like centrifugation or filtration. The clarified cell supernatant was applied to a protein A column equilibrated in DPBS pH 7.4 (Sigma/Aldrich). The column was washed with ten column volumes of DPBS pH 7.4+500 mM sodium chloride. PDGFRbeta-Fc fusion proteins were eluted in 50 mM sodium acetate pH 3.5+500 mM sodium chloride. PDGFRbeta-Fc fusion proteins were further purified employing a size exclusion chromatography step on a Superdex 200 column (GE Healthcare) in DPBS pH 7.4.

### Example 2

### BiaCore

Binding affinities (KD values) of PDGFRbeta-Fc fusion proteins were measured by using surface plasmon resonance assays. Experiments were performed using a Biacore T200 instrument (GE Healthcare Biacore, Inc.) with Series S Sensor Chips CM5 (GE Healthcare Biacore, Inc.). Binding assays were carried out at 25° C. with assay buffer HBS-EP+ supplemented with BSA (Sigma) and NaN3 (10 mM HEPES pH 7.4, 500 nM NaCl, 1 mg/ml BSA, 0.05% SP20, 0.05% NaN3). Fc fusions were captured with an anti-hIgG capture antibody covalently immobilized to the chip surface via amine coupling chemistry. Reagents for amine coupling (1-ethyl-3-(3-dimethylaminopropyl)carbodiimide hydrochloride (EDC), N-hydroxysuccinimide (NHS), ethanolamine-HCl pH 8.5) were used from the Amine Coupling Kit (GE Healthcare, product code BR-1000-50). anti-hIgG capture antibody and immobilization buffer (10 mM sodium acetate pH 5.0) was used from the Human Antibody Capture Kit (GE Healthcare, BR-1008-39). The sensor chip surface was activated with a freshly prepared solution of 0.2 M EDC and 0.05 M NHS passed over the chip surface for 420 s at a flow rate of 10 μl/min, followed by an injection of anti-hIgG capture antibody (dissolved to 25 μg/ml in immobilization buffer) for 180 s at a flow rate of 5 μl/min. Excess of activated groups were blocked with a 1 molar solution of ethanolamine injected at a flow rate of 10 μl/min for 420 s.

hPDGF-AA-AB-BB-CC-DD obtained from R&D Systems were used as analyte to determine K_{D}s. To generate a titration curve various concentrations between 3.9 and 500 nM of PDGF-AA (Recombiant Human PDGF-AA, CF, Cat. No. 221-AA-010), 1.6 and 200 nM of PDGF-AB (Recombiant Human PDGF-AB, CF, Cat. No. 222-AB-010), 0.01 and 1.5 nM of PDGF BB (Recombinant Human PDGF-BB, CF, R&D Systems, Cat. No. 220-BB-010), 3.9 and 500 nM of PDGF-CC (Recombinant Human PDGF-CC, CF, R&D Systems, Cat. No. 1687-CC-025/CF) and 0.01 and 1.5 nM PDGF DD (Recombinant Human PDGF-DD; R&D Systems; Cat. No. 1159-SB-025/CF) in assay buffer (see above) were injected over captured Fc-fusion proteins at a flow rate of 60 μl/min for 3 minutes and the dissociation was monitored for 10 minutes.

Obtained sensorgrams were double-referenced, i.e. in-line reference cell correction followed by buffer sample subtraction. K_{D} values for PDGF BB and DD were calculated based on the ratio of dissociation (k_{d}) and association (kₐ) rate constants which were obtained by globally fitting sensorgrams with a first order 1:1 Langmuir binding model. Data for PDGF AB were evaluated by a steady-state affinity plot. No binding of the constructs to PDGF-AA and -CC was observed. K_{D} values for PDGF-AB were determined to be in the range of ˜100 nM+/−50 nM. K_{D} values of the different constructs to PDGF-BB and -DD are listed in table 1 below.

TABLE 1
K_{D} values of different PDGFRbeta Fc constructs
and the respective ligands BB and DD, [nM].

| | |
|---|---|
| PDGF BB | PDGF DD |
| (1.5-0.01 nM) | (1.5-0.01 nM) |

| | |
|---|---|
| compound | KD [nM] |

| | | |
|---|---|---|
| No linker | 0.055 | 0.035 |
| — | | |
| EPKSC | 0.045 | 0.073 |
| EPKSS | 0.031 | 0.066 |
| GGGGG | 0.019 | 0.054 |
| 1xGGGGS | 0.017 | 0.057 |
| 2xGGGGS | 0.008 | 0.043 |
| 3xGGGGS | 0.007 | 0.025 |
| 4xGGGGS | 0.005 | 0.016 |
| 5xGGGGS | 0.004 | 0.012 |
| 6xGGGGS | 0.005 | 0.011 |
| 7xGGGGS | 0.006 | 0.012 |

In summary, PDGFRbeta-Fc-fusion proteins without linkers bound with affinities of 0.055 nM to PDGF-BB and 0.035 nM to PDGF-DD respectively. Addition of linkers increased the affinity of binding to PDGF-BB. Affinities increased with linkers equal or longer than 2×GGGGS to 0.004-0.008 nM (difference within the variability of the measurement). For PDGF-DD affinities increased with linkers equal or longer than 3×GGGGS as well up to 0.011-0.025 nM. The affinities increased with linker length up to 4×GGGGS linker (SEQ ID NO: 10) by a factor of 11 for PDGF-BB. Longer linkers did not result in higher affinities.

### Example 3

### Cell-Based Assays to Determine In Vitro Potency of PDGFRbeta-Fc Fusion Proteins

PDGFRbeta-Fc fusion proteins have been characterized in vitro using Normal Human Dermal Fibroblasts (NHDF) (NHDF juvenile foreskin, Promocell, Catalog Number: C-12300, cultivated according to provider's manual, (http://www.promocell.com/fileadmin/promocell/PDF/C-12300.pdf) cell-based assays in which two downstream signaling events after stimulation with human PDGF-BB (Recombinant Human PDGF-BB, CF, R&D Systems, Cat. No. 220-BB-010) were assessed:
pPDGFRbeta Tyr751 phosphorylation (Meso Scale Phospho PDGFR-beta (Tyr751) Assay Whole Cell Lysate Kit K150DVD-1)
pAKT Ser473 phosphorylation (Meso Scale Phospho (Ser473) Total AKT Assay Whole Cell Lysate Kit K15100D-1)

NHDFs were starved for 3 h with serum-free culture medium, stimulated for 30 min with 6.3 ng/ml (for pAKT assays) and 50 ng/ml (for pPDGFRbeta assays) PDGF-BB (R&D Systems, Recombinant Human PDGF-BB, CF; 220-BB-010), which was pre-incubated with varying concentrations between 4.0×10⁻⁸ to 8.6×10⁻¹² M of the different PDGFRbeta-Fc fusion proteins for 30 min at room temperature, followed by cell lysis using the manufacturer provided lysis buffer for further analysis. The sample measurement and the data analysis were performed on the Meso Scale platform according to manufacturer's instructions.

TABLE 2
IC₅₀ values [M] of different PDGFRbeta-Fc fusion
proteins in PDGFRbeta and pAKT MesoScale assays.

| | |
|---|---|
| pPDGFRβ | pAKT |

IC₅₀ [M]

| | | |
|---|---|---|
| no linker | 3.18E−09 | 1.03E−08 |
| 1x GGGGS | 1.61E−09 | 6.42E−10 |
| 2x GGGGS | 1.35E−09 | 2.90E−10 |
| 3x GGGGS | 3.22E−10 | 1.22E−10 |
| 4x GGGGS | 2.51E−10 | 9.36E−11 |
| 5x GGGGS | 3.33E−10 | 7.98E−11 |
| 6x GGGGS | 2.83E−10 | 9.28E−11 |
| 7x GGGGS | 3.11E−10 | 8.92E−11 |

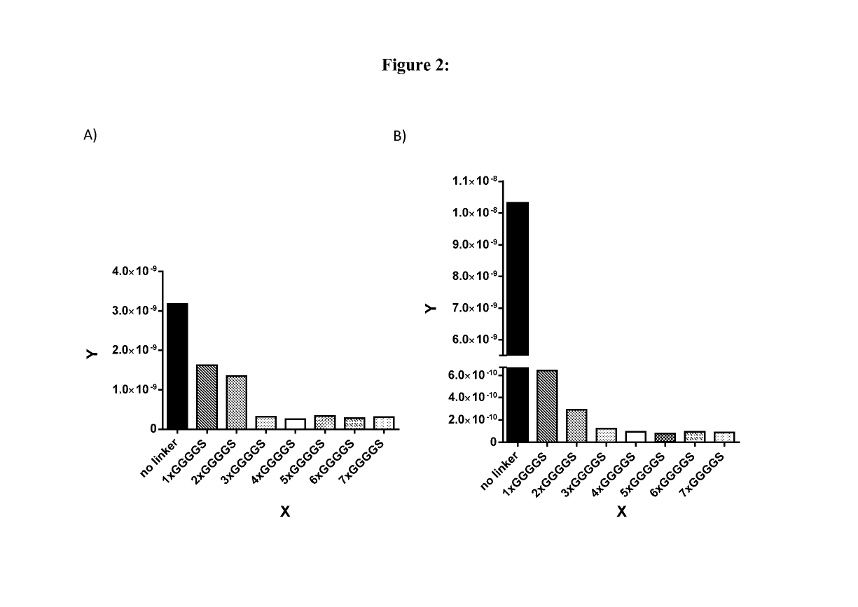

Table 2 shows that in the pPDGFRbeta assay IC50 values declined from 3.18×10⁻⁹ M for the variant containing no linker to 2.51×10⁻¹⁹ M for the 4×GGGGS linker, corresponding to a factor of 12.7 (FIG. 2A). In the pAKT assay the reduction of the IC₅₀ values by the addition of a linker was even more pronounced by a reduction 129-fold lower IC₅₀ value for the 5×GGGGS linker variant (FIG. 2B). The IC₅₀ values for the variants carrying a linker of >3×GGGGS were not statistically different from each other. This increase in in vitro potency was unexpectedly high when compared to the increases seen in binding affinity in the BIAcore assay.

### Example 4

### Animal Models

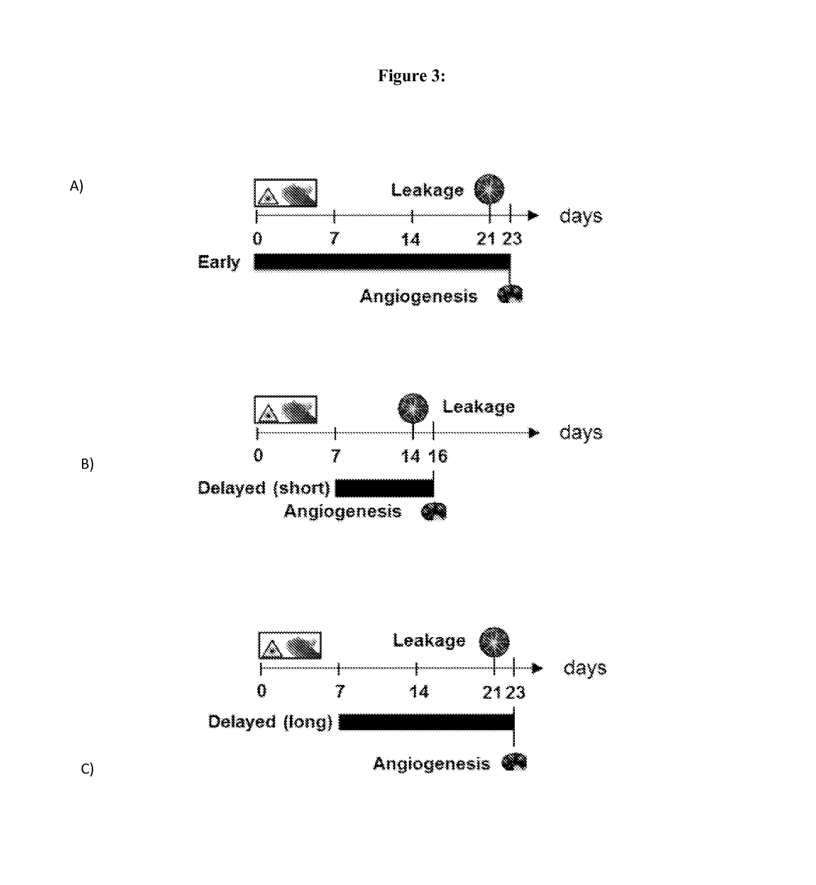

The rat laser Choroidal-Neovascularization (CNV) model was used for the in vivo pharmacological characterization of PDGFRbeta-Fc fusion proteins. Ingrowth of vessels from the choroidea into the retina was induced by applying a laser burn to the choroid layer. Three different treatment schedules were used to assess the efficacy of therapeutic agents on neovascularization and vascular leakage (FIG. 3). In the early treatment protocol (FIG. 3A) the therapeutic agent was administered intravitreally by a single injection of 2 μl of a PBS solution of the test compound one day after the laser burn. Animals were sacrificed on day 23 after the laser procedure. Two days prior to the end of the experiment a fluorescence angiography was performed in order to assess the vascular leakage at the lesion site by scoring the amount of fluorescence emanating from the newly formed vessels. The vascular leakage score was determined using a fluorescence fundus camera (Kowe). After anaesthesia and pupillary dilation, 10% sodium fluorescein dye was injected subcutaneously, and images were recorded 2 and 10 min after dye injection. The vascular leakage of the fluorescein on the angiograms was evaluated by three different examiners who were blinded for group allocation (test compound versus vehicle), and scored with 0 (no leakage) to 3 (strongly stained). The retinae were then collected on day 23 of the experiment for immunohistochemical analysis of the vessels to allow quantification of the neovascular area at the laser-induced lesion. The neovascular area is determined as follows: On day 23, animals were sacrificed, and eyes were harvested and fixed in 4% paraformaldehyde solution for 1 hour at room temperature. After washing, the retina was carefully peeled, washed, blocked and stained with a FITC-isolectin B4 antibody in order to visualize the vasculature. Then, the sclera-choroids were flat-mounted and examined under a fluorescence microscope (Keyence Biozero) at 488 nm excitation wavelength. The area (in μm²) of choroidal neovascularization was measured using ImageTool software.

In the delayed treatment setting intravitreal dosing was performed on day 7 after the laser burn. The protocol had two variants with either 9 (short, FIG. 3B) or 16 days (long, FIG. 3C) duration. Evaluation of the animals in the course of the experiment was identical to the early treatment protocol with the exception of the short delayed treatment protocol. Here, a fluorescence angiography was performed prior to treatment on day 7. The vascular leakage measured on day 7 served as baseline value for the assessment of vascular leakage on day 14. Therefore, changes in leakage are given in % of baseline in this experimental setting.

### 4.1 Rat Laser CNV—Early Treatment Setting

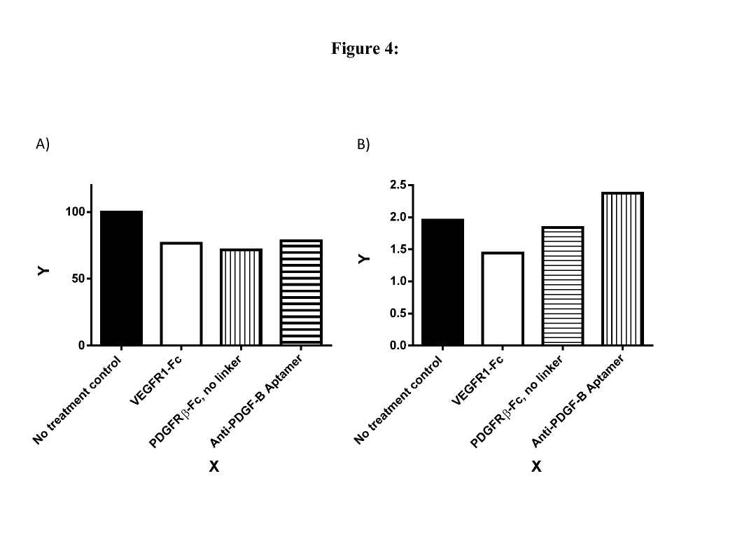

The linker-less PDGFRbeta-Fc fusion protein was compared to a VEGFR1-Fc fusion protein (R&D Systems, Recombinant Human VEGF R1/Flt-1 Fc Chimera, CF, 321-FL-050/CF) and a pegylated PDGF-B aptamer corresponding to a previously published compound (Akiyama et al., J Cell Physiol 2006; 207:407-412). This aptamer is similar to the currently clinically tested PDGF-B aptamer Fovista. In the early treatment setting where anti-VEGF strategies generally show good efficacy, the linker-less PDGFRbeta-Fc fusion protein and the PDGF-B aptamer had similar efficacy as anti-VEGF as stand-alone treatments in reducing neovascularization (FIG. 4A). In contrast, while anti-VEGF shows a significant reduction in vascular leakage, the linker-less PDGFRbeta-Fc construct has no effect on leakage in this setting (FIG. 4B). The PDGF-B aptamer on the other hand increases vascular leakage significantly compared to the anti-VEGF and the linker-less PDGFRbeta-Fc fusion protein treatment group (FIG. 4B).

Without being bound by theory, these data imply that inhibition of the PDGF signaling pathway with a FDGFRbeta-Fc approach has the potential to reduce neovascularization in wet AMD while no potentially adverse increase in vascular leakage is observed.

### 4.2. Rat Laser CNV—Delayed Treatment Setting

### 4.2.1 Short Delayed Treatment Setting

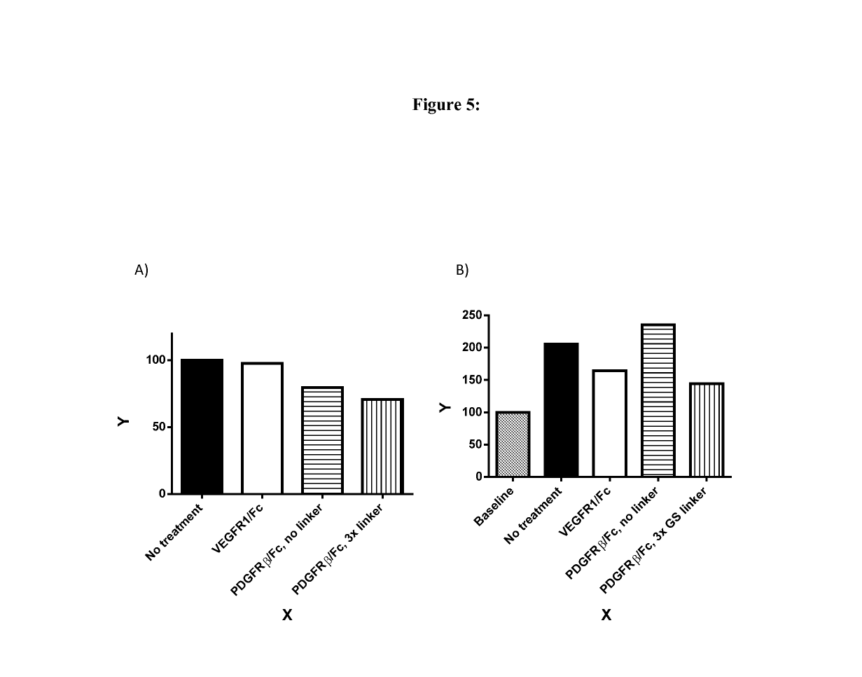

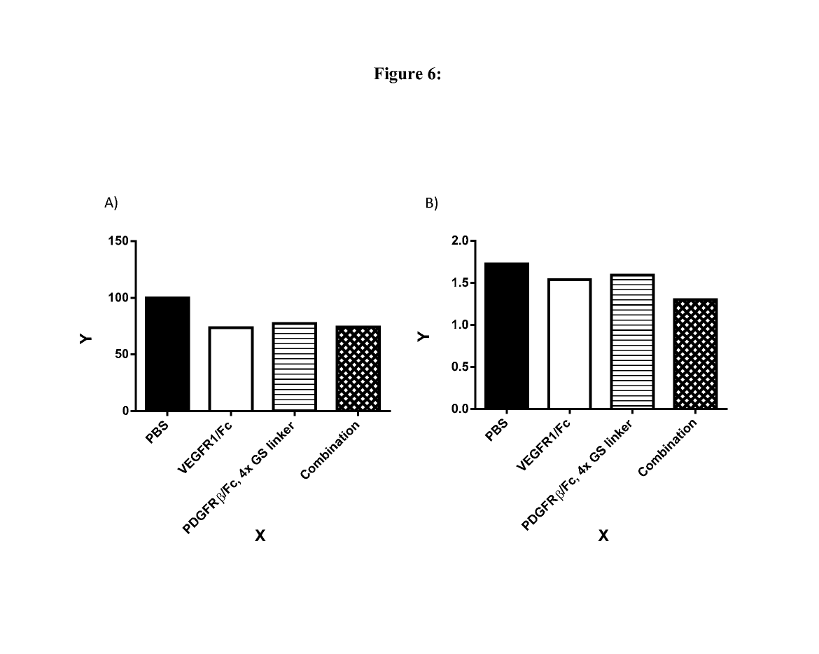

In the short delayed treatment setting the effect of anti-VEGF (VEGFR1-Fc fusion protein (R&D Recombinant Human VEGF R1/Flt-1 Fc Chimera, CF, 321-FL-050/CF)) on neovascularization was reduced or even absent. In this setting, both the linker-less PDGFRbeta-Fc fusion protein and the 3×GGGGS linker PDGFRbeta-Fc fusion protein showed a significant reduction in neovascularization as stand-alone regimens compared to the no treatment control and the VEGFR1-Fc fusion protein (FIG. 5A). In this protocol there was also a reduction in vascular leakage that became statistically significant surprisingly for the 3×GGGGS PDGFRbeta-Fc fusion protein and was more pronounced in both experiments than the reduction seen with the VEGFR1-Fc fusion protein (FIG. 5B). The 3×GGGGS linker PDGFRbeta-Fc fusion protein reduces neovascularization to a greater extent than the linker-less variant and even reduces vascular leakage in this setting, adding an unexpected potential therapeutic benefit over current agents targeting PDGF signaling.

### 4.2.2 Long Delayed Treatment Setting

The PDGFRbeta-Fc fusion protein with a 4×GGGGS linker was tested in the delayed treatment protocol with long treatment duration as stand-alone and in combination with a VEGFR1-Fc fusion protein (R&D Systems, Recombinant Human VEGF R1/Flt-1 Fc Chimera, CF, 321-FL-050/CF). The 4×GGGGS linker PDGFRbeta-Fc fusion protein confirmed the findings from the short delayed treatment protocol with the linker-less PDGFRbeta-Fc fusion protein and the 3×GGGGS linker PDGFRbeta fusion protein on the reduction of neovascularization. An additive effect on neovascularisation in combination with anti-VEGF was not observed, which, without being bound by theory, could be due to the ceiling effect mentioned above (FIG. 6A).

Vascular leakage is not significantly reduced by either the VEGFR1-Fc fusion protein or the 4×GGGGS linker PDGFRbeta-Fc fusion protein alone in this experiment. The combination of the VEGFR1-Fc fusion protein and the 4×GGGGS linker PDGFRbeta-Fc, fusion protein however, resulted in a significant reduction of vascular leakage, demonstrating the benefit of such a combination (FIG. 6B).

### Example 5

### Pharmacokinetics and Drug Metabolism

Pharmacokinetic data within this application is used to describe characteristics of a class of receptor-Fc-fusion proteins targeting PDGF ligands.

### PK Assay (ELISA)

For quantification of “total” PDGFRbeta-Fc proteins and Lucentis® as experimental reference, ligand binding assays were used with a lower limit of quantification of 78-156 μg/L in plasma and eye compartment preparations.

For quantification of “total” PDGFRbeta-Fc proteins in rabbit plasma Greiner 96-well plates were coated with AffiniPure Goat Anti-Human IgG, Fcγ Fragment Specific (109-005-008; Jackson Immunotech) for capturing. BSA was used as a blocking agent. After incubation of samples, detection was performed with HRP-conjugated AffiniPure Donkey Anti-Human IgG, Fcγ Fragment Specific (709-036-098; Jackson Immunotech) after the addition of OPD (o-phenylenediamine dihydrochloride, PI-34006; Thermo Pierce) as substrate.

For quantification of Lucentis® in PK studies, Greiner 96-well plates were coated with donkey anti-human IgG Ab, H+L specific, (709-006-149; Jackson Immunotech). BSA was used as blocking agent. After incubation of samples, detection was performed with HRP-conjugated goat anti-human IgG Ab, (Fab)2 fragment specific, (109-035-006; Jackson Immunotech) after the addition of OPD (Thermo PI-34006; Thermo Pierce) as substrate.

### Example 6

### In Vivo PK in Healthy NZW Rabbits

### 6.1: Comparison of Different Eye Compartments

Pharmacokinetics of the linker-less PDGFRbeta-Fc, the 3×GGGGS and the 4×GGGGS linker PDGFRbeta-Fc fusion proteins were investigated in healthy female New Zealand White rabbits after single intravitreal co-injection of 625 μg/eye and 250 μg/eye Lucentis® as experimental reference. 100% PBS was used as 2-in-1 formulation for compound and reference. Plasma, aqueous and vitreous humor, as well as the retinal compartment of administered eyes were analyzed.

The percentage exposure (area under the curve, AUC, a measure for the amount of substance present in the analyzed compartment) of the compounds was analyzed within the three eye compartments, as well as plasma samples. The 4×GGGGS linker PDGFRbeta-Fc fusion protein, as representative for all tested PDGFRbeta-Fc fusion proteins, as well as Lucentis® show similar results (see Table 3). The partial distribution, calculated based on AUC values of the single compartments, reveals the majority (˜90%) of administered compound within the vitreous, whereas only a minor part of the compound is located in the aqueous humor (˜8%) and the retinal compartment (˜2%). Neither Lucentis® nor any of the PDGFRbeta-Fc proteins were detected in rabbit plasma.

TABLE 3
Distribution of different PDGFRbeta-Fc fusion
protein variants in different eye compartments

| | | vitreous | aqueous | retinal |
|---|---|---|---|---|
| | | humor | humor | compartment |
| no linker | % AUC_{eye} | 90 | 8 | 2 |
| 4x GGGGS | % AUC_{eye} | 89 | 9 | 2 |
| 3x GGGGS | % AUC_{eye} | 89 | 9 | 2 |

Additionally, the 4×GGGGS linker PDGFRbeta-Fc fusion protein, as well as all other tested PDGFRbeta-Fc fusion protein variants and Lucentis® show similar, in parallel declining elimination profiles within all analyzed eye compartments.

Regarding these results, vitreous humor as compartment of interest, was used as sole matrix for consideration in all following discussions.

### 6.2: Comparison of Pharmacokinetics of Different PDGFRbeta-Fc Fusion Protein Versus Lucentis®

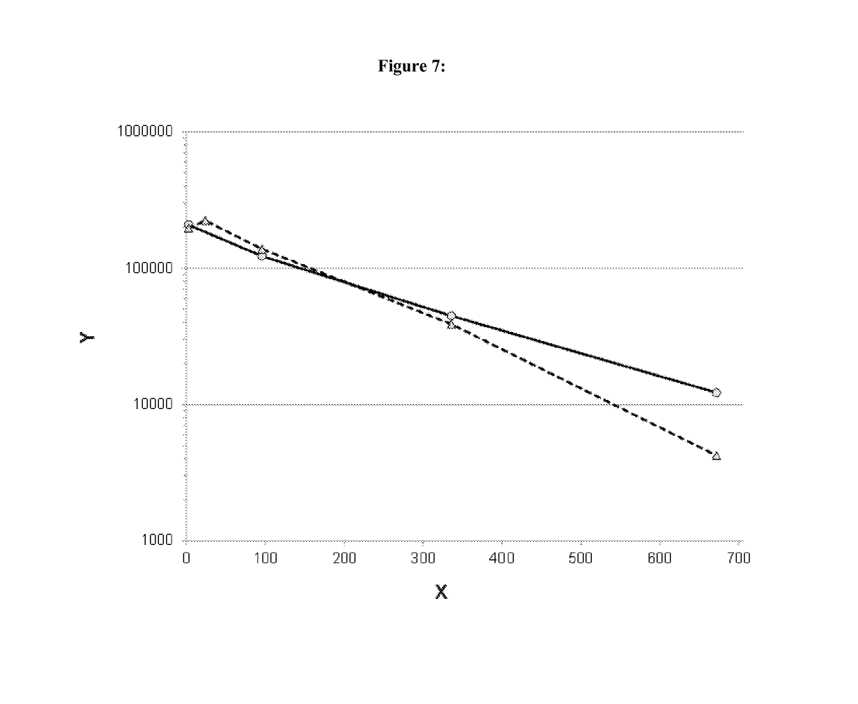

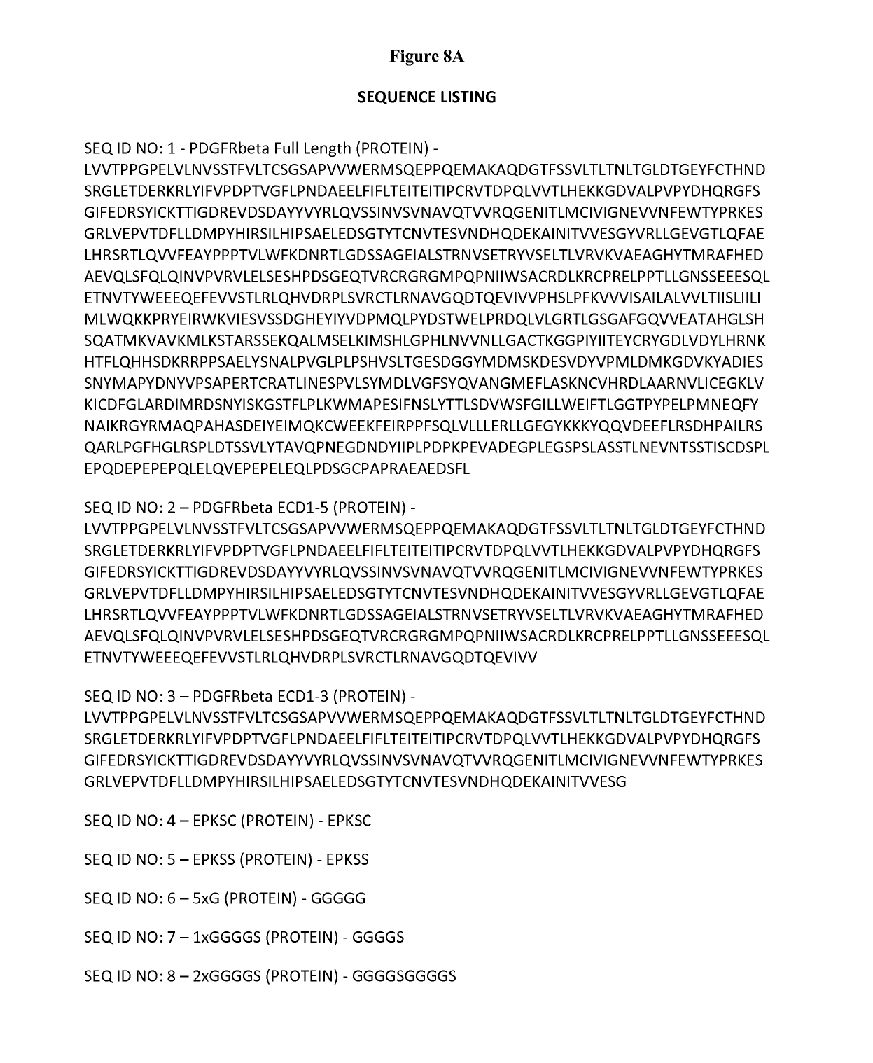

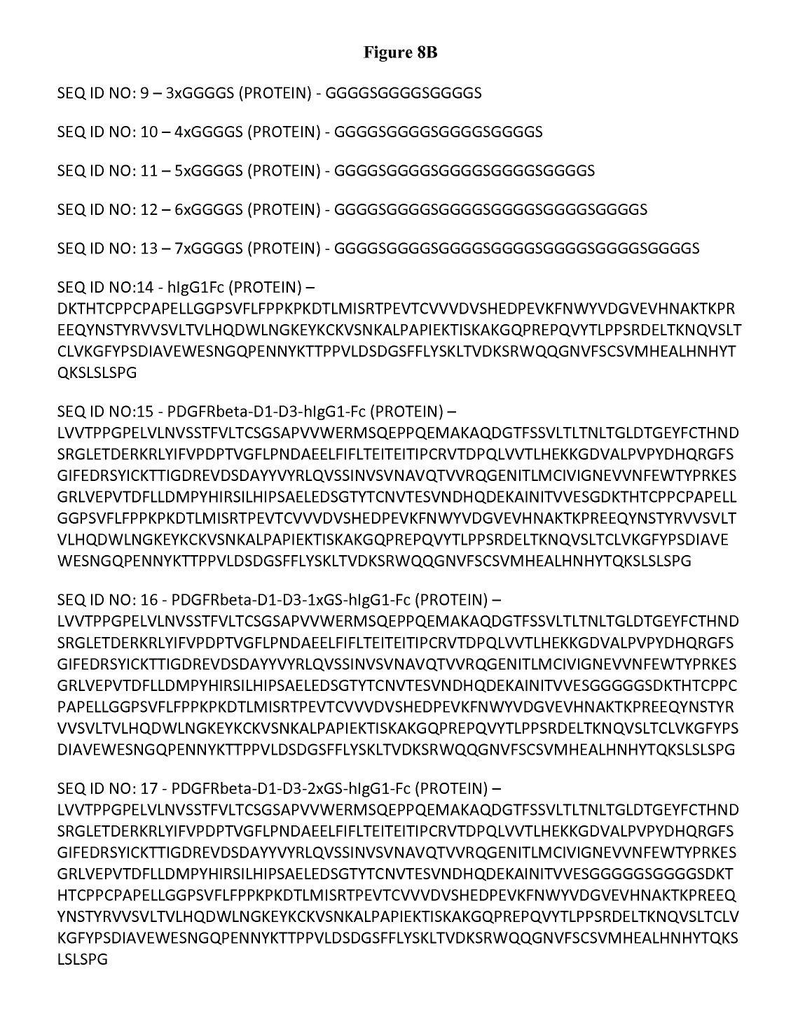

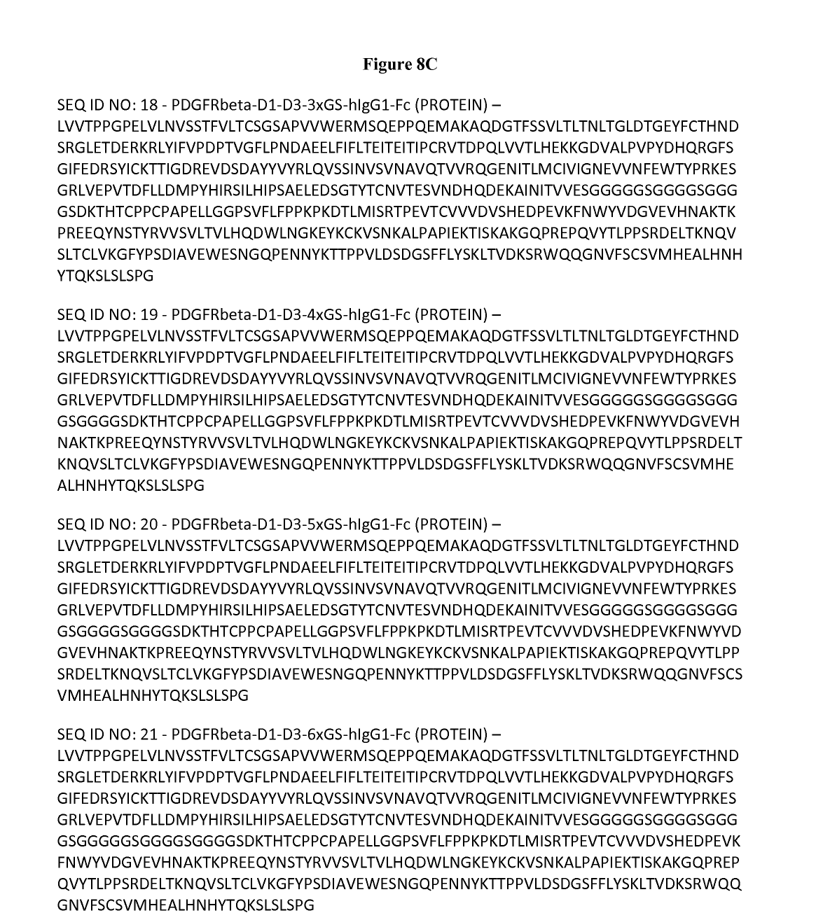

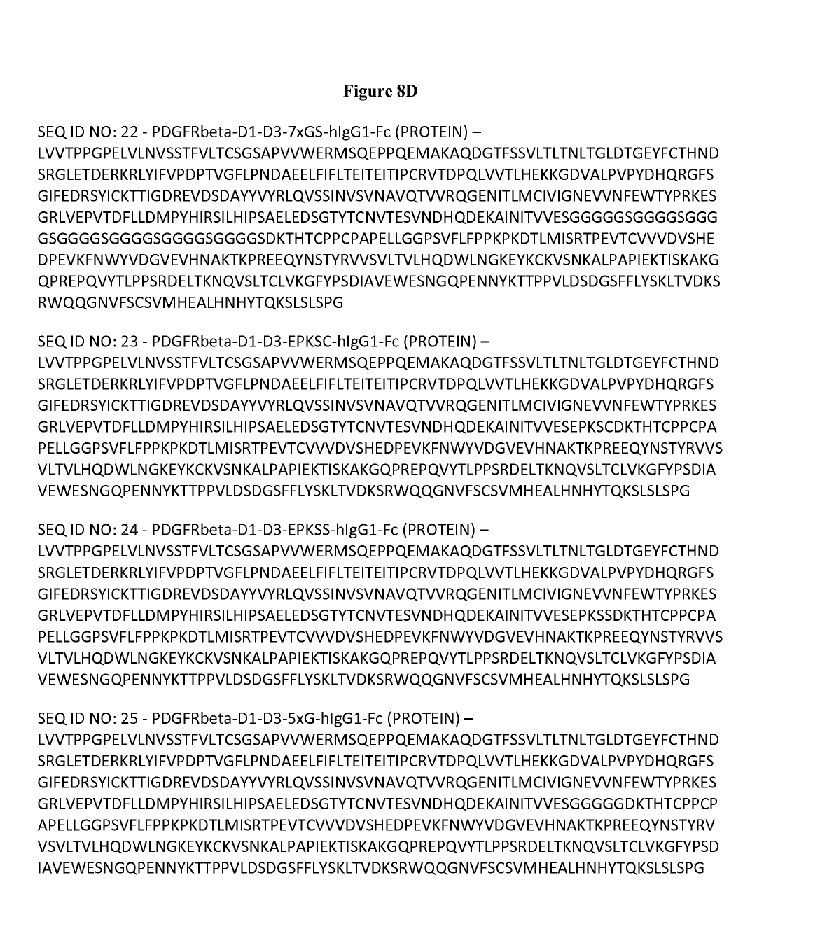

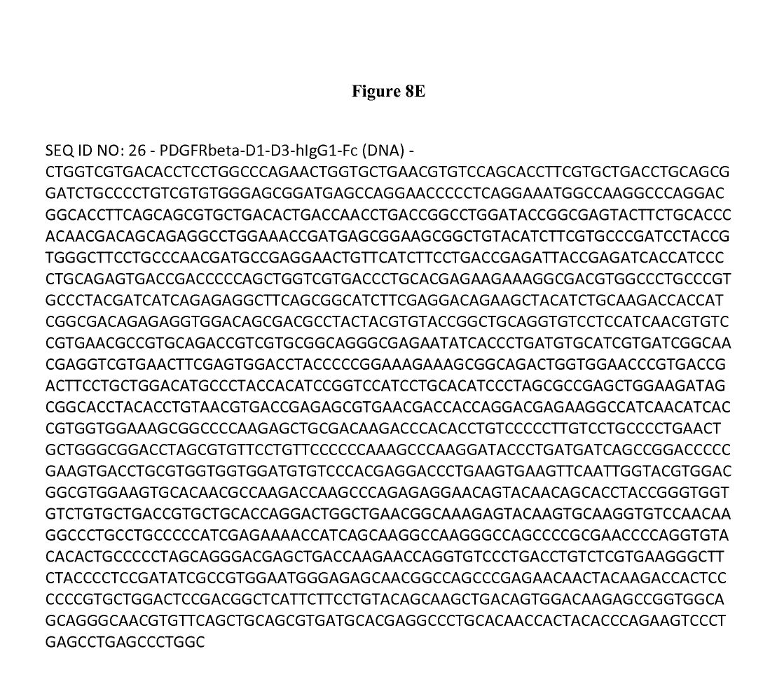

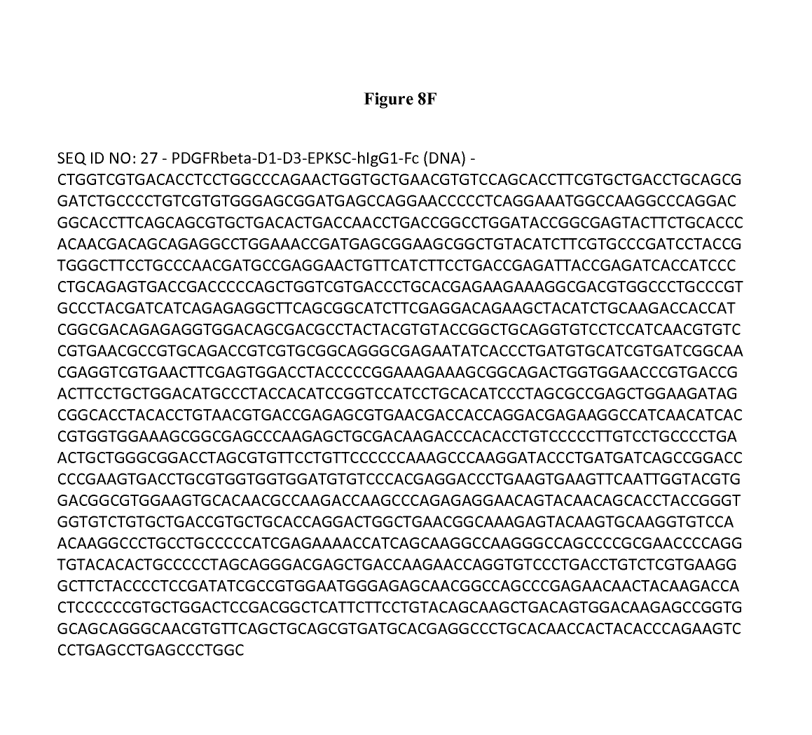

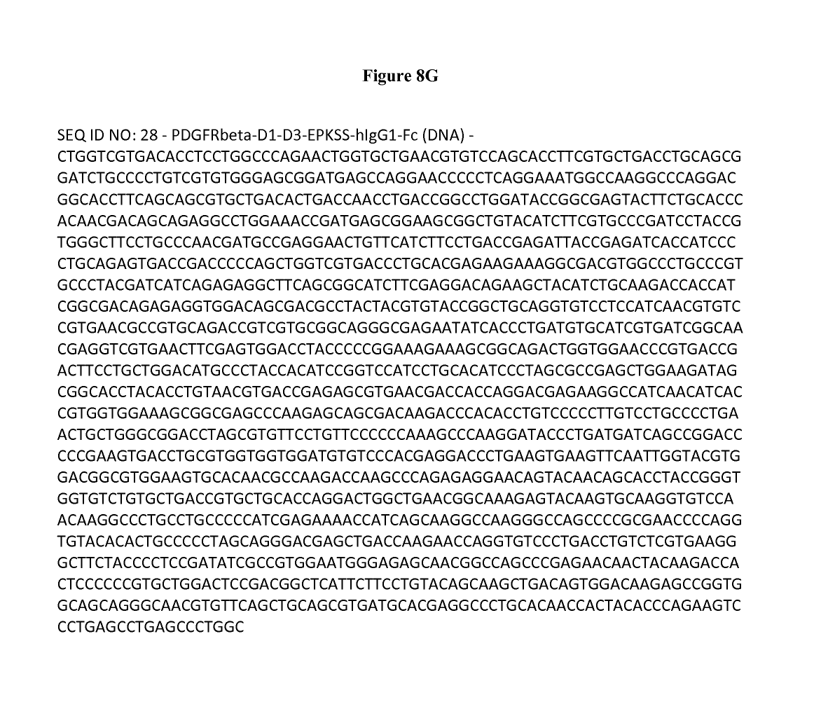

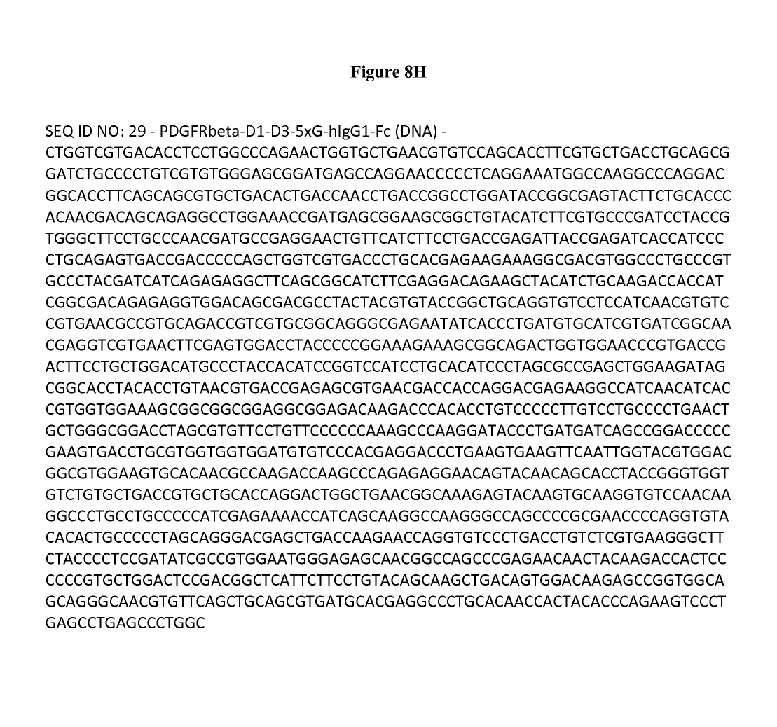

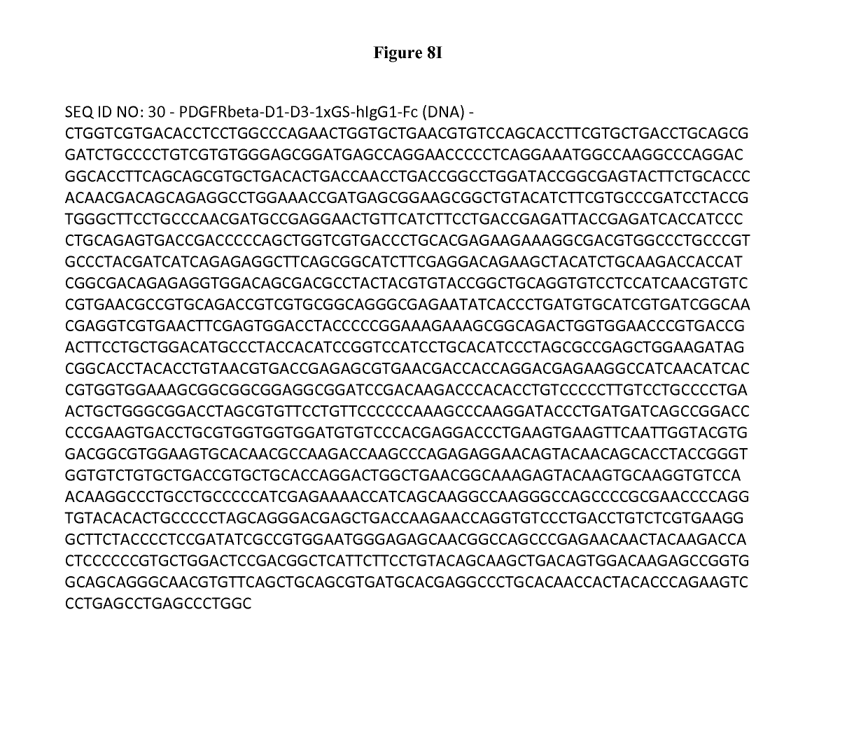

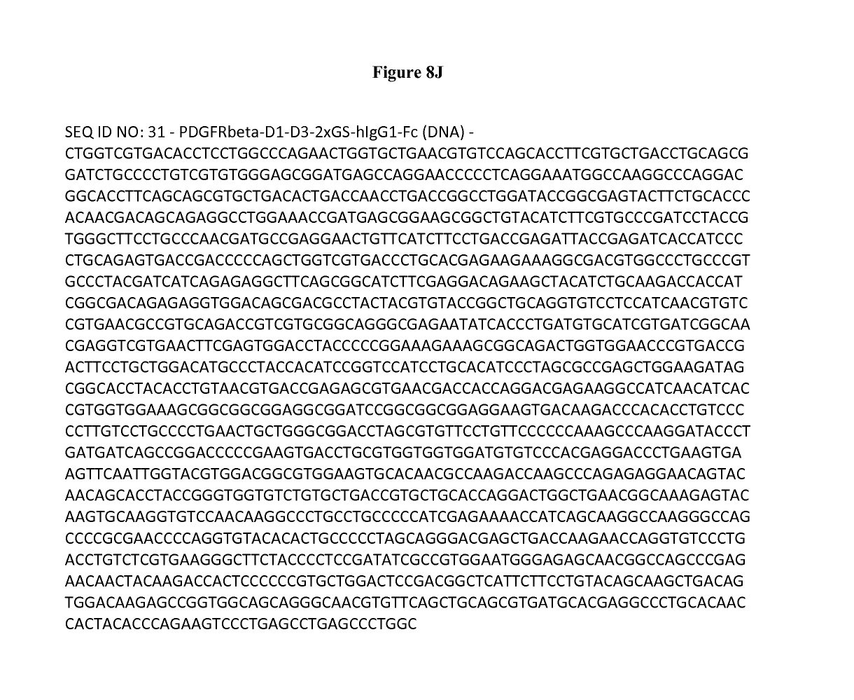

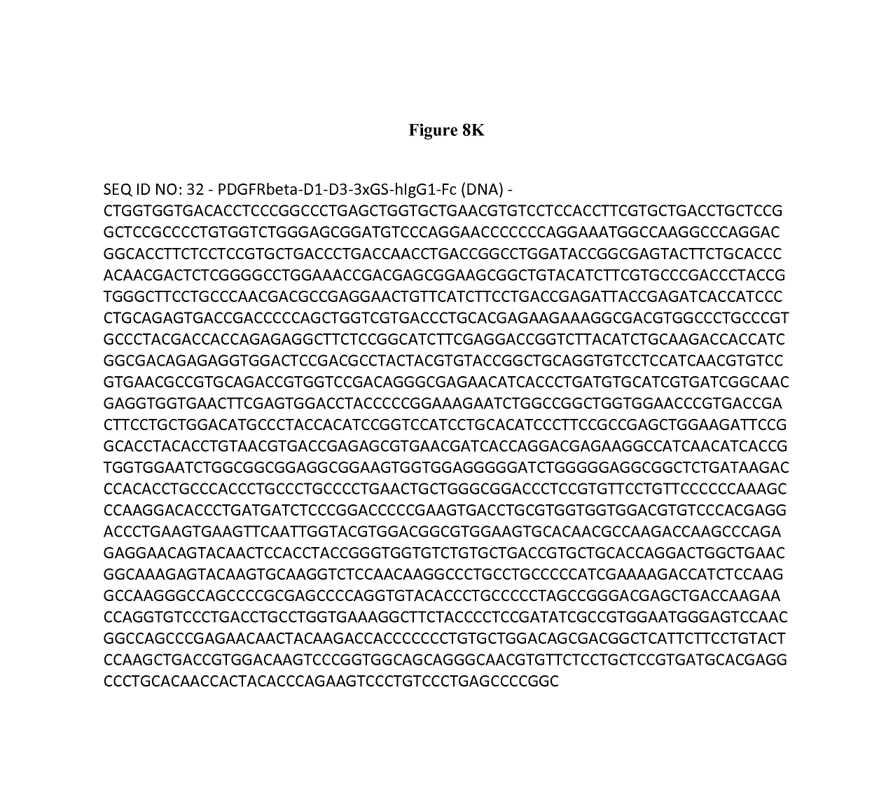

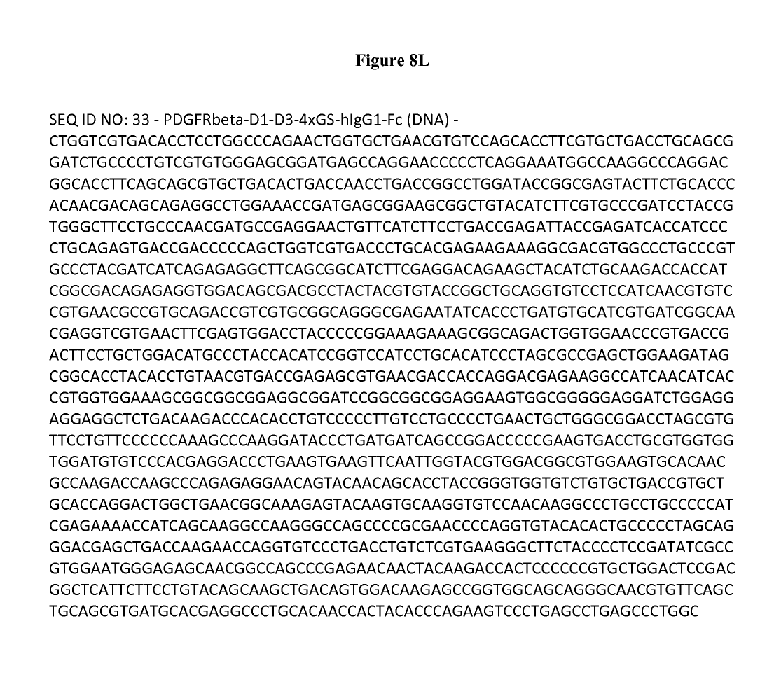

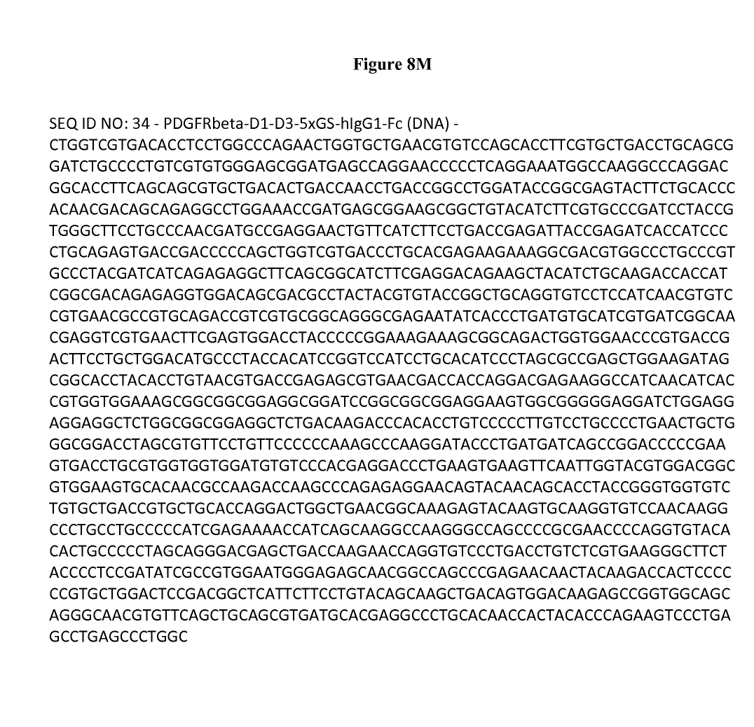

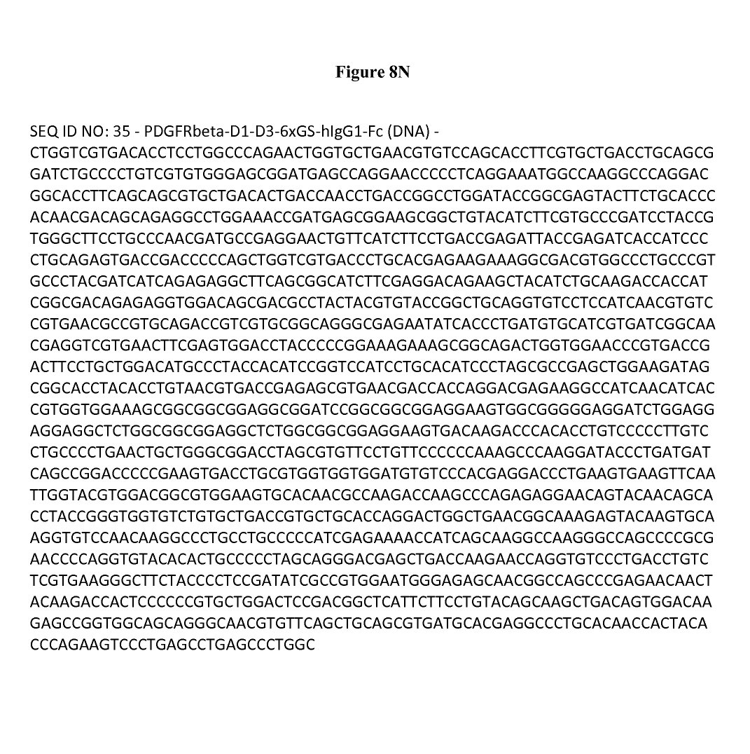

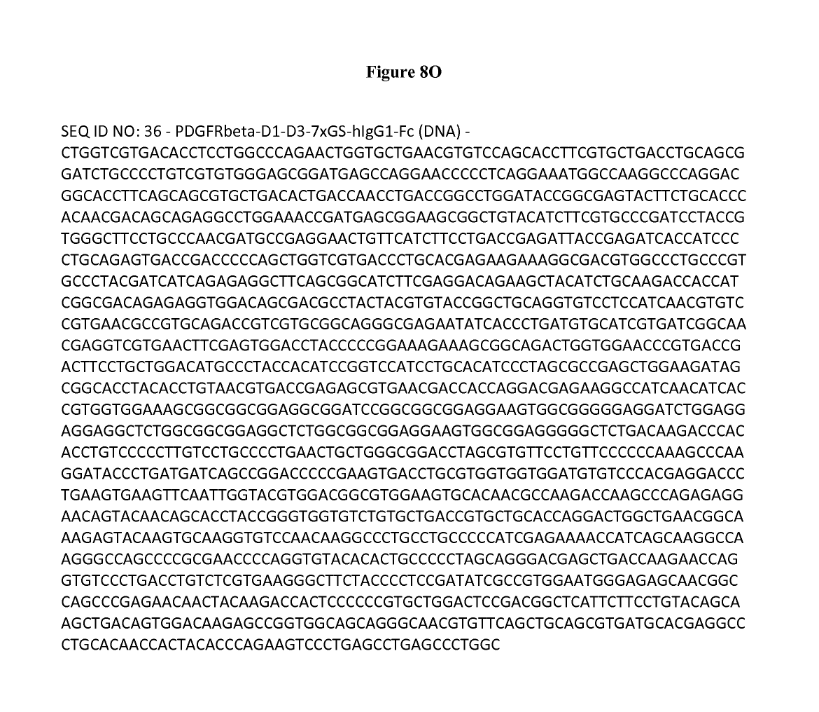

Concerning vitreous residence times Lucentis® revealed a Mean Residence Time (MRT) of 5.1 days and a terminal half-life of 3.3 days (FIG. 7 and Table 4), both clearly in line with published data for Lucentis® (MRT=4.0 days and t½=2.9 days). The linker-less PDGFRbeta-Fc fusion protein shows a longer vitreal terminal half-life of about 4.8 days and an MRT of 7.3 days after administration of 625 μg into the vitreous. 3 hours after administration, 68% of the linker-less PDGFRbeta-Fc fusion protein was found within the total eye. At least 62% of it was recovered from the vitreous. These data were expected given the published characteristics of Eylea™ (aflibercept), another clinically used Fc-fusion protein (cf. Table 4). The comparison between human and rabbit intravitreal half-lives of Eylea also validates the rabbit as a model organism for making predictions of human pharmacokinetic properties of Fc-fusion proteins.

The 3×GGGGS and 4×GGGGS PDGFRbeta-Fc fusion protein variants both showed a significant and unexpected increase compared to the linker-less variant in intravitreal terminal half-life to 7.13 and 7.21 days.

PK parameters were calculated from FIG. 7 (solid line—4×GGGGS linker PDGFRbeta-Fc fusion protein; dashed line—linker-less PDGFRbeta-Fc fusion protein variant).

TABLE 4
Comparison of intravitreal half-lives after intravitreal injection of PDGFRbeta-Fc
fusion proteins and anti-VEGF therapeutics in rabbit and human.

| | PDGFRbeta- | PDGFRbeta- | PDGFRbeta- | | | | |
|---|---|---|---|---|---|---|---|
| | Fc w/o | Fc 3xGGGGS | Fc 4xGGGGS | Lucentis ™ | Lucentis ™ | Eylea ™ | Eylea ™ |
| | linker | linker | linker | ref. data | lit. data | lit. data | lit. data |
| species | rabbit | rabbit | rabbit | rabbit | rabbit | rabbit | human |
| t_{1/2} [d] | 4.75 | 7.13 | 7.21 | 3.33 | 2.88 (1) | 4.58 (2) | 4.79 (3) |

Intravitreal administration of 625 μg/eye PDGFRbeta-Fc to healthy female NZW rabbits, co- administered with 250 μg/eye Lucentis ®. ref. data (reference data) - data measured for calibration with published data, lit. data (literature data) - published data, references see below.
(1) Bakri S. J. et al., Ophthalmology 114(12): 2179-2182, December 2007
(2) Christoforidis J. B. et al., Curr Eye Res 37(12): 1171-1174, Epub Sep 19th 2012
(3) http://www.ema.europa.eu/docs/en_GB/document_library/EPAR_-_Public_assessment_report/human/002392/WC500135744.pdf

Due to its molecular structure and its pharmacological properties, a similar pharmacokinetic profile for the linker-less PDGFRbeta-Fc fusion protein and the PDGFRbeta-Fc fusion protein with different linker length was expected. Surprisingly, however, the PDGFRbeta-Fc fusion protein with a 4×GGGGS linker showed an extended intravitreal halflife over the linker-less PDGFRbeta-Fc fusion protein. When compared to published data on the intravitreal half-life of Eylea™, these data were also unexpected.

This property is highly desirable to enable a reduction in intravitreal dosing frequency, which reduces the risk of adverse events associated with intravitreal application such as infection, the physical and psychological burden and the cost of more frequent intravitreal injections for the patient.

1. A PDGFRbeta-Fc fusion protein consisting essentially of:
(i) a polypeptide consisting essentially of extracellular domains D1-D3 of Platelet-Derived Growth Factor Receptor Beta (PDGFRbeta);
(ii) a Fc domain, and
(iii) a linker that is selected from the group consisting of SEQ ID NOs: 10-13;
wherein the C-terminal amino acid of the extracellular domain D3 is connected to the N-terminal amino acid of the Fc domain by means of the linker.

2. The PDGFRbeta-Fc fusion protein of claim 1, wherein the polypeptide consists essentially of an amino acid sequence that is at least 95% identical to the amino acid sequence as set forth in SEQ ID NO: 3.

3. The PDGFRbeta-Fc fusion protein of claim 1, wherein the polypeptide consists essentially of an amino acid sequence as set forth in SEQ ID NO: 3.

4. The PDGFRbeta-Fc fusion protein of claim 1, wherein the Fc domain consists essentially of the Fc domain of a human IgG1.

5. The PDGFRbeta-Fc fusion protein of claim 2, wherein the Fc domain consists essentially of the Fc domain of a human IgG1.

6. The PDGFRbeta-Fc fusion protein of claim 3, wherein the Fc domain consists essentially of the Fc domain of a human IgG1.

7. The PDGFRbeta-Fc fusion protein of claim 5, wherein the linker consists essentially of the amino acid sequence of SEQ ID NO: 10.

8. The PDGFRbeta-Fc fusion protein of claim 6, wherein the PDGFRbeta-Fc fusion protein consists essentially of the amino acid sequence of SEQ ID NO: 19.

9. The PDGFRbeta-Fc fusion protein of claim 5, wherein said fusion protein binds one or more of the PDGF ligands -BB, -DD, and -AB with a K_{D} of less than 125 pM.

10. The PDGFRbeta-Fc fusion protein of claim 5, wherein said fusion protein binds the PDGF ligand -BB with a K_{D} of less than 30 pM.

11. The PDGFRbeta-Fc fusion protein of claim 5, wherein said fusion protein binds the PDGF ligands -BB and -DD with a K_{D} of less than 30 pM.

12. The PDGFRbeta-Fc fusion protein of claim 5, wherein said fusion protein has an IC₅₀ for phosphorylation of Protein Kinase B (AKT) that is at least 10-fold lower than that of a linker-less PDGFRbeta-Fc fusion protein in a PDGF-BB-mediated phosphorylation assay.

13. The PDGFRbeta-Fc fusion protein of claim 5, wherein said fusion protein has an intravitreal half-life that is at least 30% longer than that of a linker-less PDGFRbeta-Fc fusion protein.

14. The PDGFRbeta-Fc fusion protein of claim 5, wherein the relative increase of in vitro potency of said fusion protein compared to the linker-less PDGFRbeta-Fc fusion protein measured as IC50 for phosphorylation of Protein Kinase B (AKT) in a PDGF-BB-mediated phosphorylation assay is at least 6 times greater than the increase in binding affinity of said fusion protein for the PDGF ligand -BB compared to the linker-less PDGFRbeta-Fc fusion protein.

15. The PDGFRbeta-Fc fusion protein of claim 8, wherein said fusion protein has an IC₅₀ for phosphorylation of Protein Kinase B (AKT) that is at least 10-fold lower than that of a linker-less PDGFRbeta-Fc fusion protein in a PDGF-BB-mediated phosphorylation assay.

16. The PDGFRbeta-Fc fusion protein of claim 8, wherein said fusion protein has an intravitreal half-life that is at least 30% longer than that of a linker-less PDGFRbeta-Fc fusion protein.

17. The PDGFRbeta-Fc fusion protein of claim 8, wherein the relative increase of in vitro potency of said fusion protein compared to the linker-less PDGFRbeta-Fc fusion protein measured as IC50 for phosphorylation of Protein Kinase B (AKT) in a PDGF-BB-mediated phosphorylation assay is at least 6 times greater than the increase in binding affinity of said fusion protein for the PDGF ligand -BB compared to the linker-less PDGFRbeta-Fc fusion protein.

18. A pharmaceutical composition comprising the PDGFRbeta-Fc fusion protein of claim 1 and at least one pharmaceutically acceptable carrier or excipient.

19. A PDGFRbeta-Fc fusion protein consisting essentially of an amino acid sequence that is at least 97% identical to an amino acid sequence of SEQ ID NO: 19, wherein said fusion protein has a linker consisting essentially of the amino acid sequence of SEQ ID NO: 10, and wherein said fusion protein binds to one or more of PDGF ligands -BB, -DD, and -AB.

20. The PDGFRbeta-Fc fusion protein of claim 19, wherein said fusion protein binds the PDGF ligand -BB with a K_{D} of less than 30 pM.

21. The PDGFRbeta-Fc fusion protein of claim 19, wherein said fusion protein binds the PDGF ligands -BB and -DD with a K_{D} of less than 30 pM.

22. The PDGFRbeta-Fc fusion protein of claim 19, wherein said fusion protein has an IC₅₀ for phosphorylation of Protein Kinase B (AKT) that is at least 10-fold lower than that of a linker-less PDGFRbeta-Fc fusion protein in a PDGF-BB-mediated phosphorylation assay.

23. The PDGFRbeta-Fc fusion protein of claim 19, wherein said fusion protein has an intravitreal half-life that is at least 30% longer than that of a linker-less PDGFRbeta-Fc fusion protein.

24. The PDGFRbeta-Fc fusion protein of claim 19, wherein the relative increase of in vitro potency of said fusion protein compared to the linker-less PDGFRbeta-Fc fusion protein measured as IC50 for phosphorylation of Protein Kinase B (AKT) in a PDGF-BB-mediated phosphorylation assay is at least 6 times greater than the increase in binding affinity of said fusion protein for the PDGF ligand -BB compared to the linker-less PDGFRbeta-Fc fusion protein.

25. A pharmaceutical composition comprising the PDGFRbeta-Fc fusion protein of claim 19 and at least one pharmaceutically acceptable carrier or excipient, wherein the PDGFRbeta-Fc fusion protein consists essentially of the amino acid sequence of SEQ ID NO: 19.

26-30. (canceled)
